(19) United States
(12) Patent Application Publication
(10) Pub. No.: US 2024/0085416 A1
(43) Pub. Date: Mar. 14, 2024

(54) USE OF MICROVIRIN IN THE IDENTIFICATION OF MYCOBACTERIUM TUBERCULOSIS MANNOSE-CAPPED LIPOARABINOMANNAN

(71) Applicants: VANDERBILT UNIVERSITY, Nashville, TN (US); UNIVERSITY OF CAPE TOWN, Cape Town (ZA)

(72) Inventors: Megan VAN DER HORST, Nashville, TN (US); David WRIGHT, Nashville, TN (US); Jonathan M. BLACKBURN, Cape Town (ZA); Leshern KARAMCHAND, Cape Town (ZA)

(21) Appl. No.: 18/253,755

(22) PCT Filed: Nov. 16, 2021
(86) PCT No.: PCT/US2021/059448

§ 371 Date: May 19, 2023

Related U.S. Application Data
(60) Provisional application No. 63/116,646, filed on Nov. 20, 2020.

(30) Foreign Application Priority Data
Apr. 23, 2021 (GB) 2105804.5

(51) Int. Cl. G01N 33/569

(52) U.S. Cl. CPC G01N 33/5695; G01N 2333/35; G01N 2405/00

(57) ABSTRACT

The present disclosure is directed to the use of microvirin-N in the detection of Mycobacterium tuberculosis infections.

### PRIORITY CLAIM

This application claims benefit of priority to U.S. Provisional Application Ser. No. 63/116,646, filed Nov. 20, 2020, and British Application Serial No. GB2105804.5, filed Apr. 23, 2021 the entire contents of which are hereby incorporated by reference.

### FEDERAL FUNDING CLAUSE

This invention was made with government support under Grants DGE-1445197 and DGE-1937963 awarded by the National Science Foundation. The government has certain rights in the invention.

Other grants that were used for funding are “A Prototype Diagnostic Device for Rapid, Sensitive and Direct Identification and Quantification of Mycobacterium tuberculosis at Point-of-Care in Low Resource Settings” from the University of Cape Town/South African Medical Research Council, National Research Foundation of South Africa (NRF) under Grant SFP150729132726, and NRF for a Research Chair Grant No. 64760.

### BACKGROUND

### 1. Field of the Disclosure

The present disclosure relates generally to the fields of medicine, infectious disease, and molecular/cell biology. More particular, the disclosure relates to the use of microvirin-N to detect mannose-capped lipoarabinomannan and Mycobacterium tuberculosis infections.

### 2. Background

Tuberculosis (TB) remains the world's leading deadliest infectious disease despite being treatable (WHO 2018). In 2018 alone, the global burden of TB was estimated to be 10 million incident cases and over 1.4 million deaths (WHO 2018). Yet, with early diagnosis and appropriate treatment, most deaths from TB can be prevented (Pai et al., 2016). One of the contributing factors to this high mortality rate is that the causative agent, Mycobacterium tuberculosis (M. tb), is the most common opportunistic infection in HIV-positive patients (MacLean et al., 2019. People infected with HIV are approximately 21 times more likely to develop active TB and have a 2 times higher fatality rate compared to people without HIV (MacLean et al., 2019; Kwan and Ernst, 2011). Reasons for comparatively poor outcomes for HIV-positive TB patients stem from late diagnosis and delays in starting antiretroviral therapy (ART) and/or TB treatment (Kwan and Ernst, 2011). In fact, the world fails to detect 36% of the estimated new cases of TB per year (WHO 2018). Conventional TB diagnostic tests rely on resource-intensive nucleic acid amplification tests, such as the Xpert MTB/RIF test, or technologies that lack specificity and sensitivity, such as sputum smear microscopy and chest radiography. Moreover, the diagnostic accuracy of these techniques is further impaired in patients with HIV coinfection, many of whom cannot produce the required sputum sample (MacLean et al., 2019; Harries and Kumar, 2018). Globally, the use of biomarker-based rapid diagnostic testing is increasing and many countries are phasing out the use of smear microscopy and chest radiography. In response to the high TB mortality in HIV-positive patients, the World Health Organization (WHO) approved the Alere Determine TB LAM Ag lateral flow assay (LFA) as a point-of-care (POC) test for people infected with HIV and with very low CD4 counts (<100 CD4 cells/μL) (WHO 2018).

The Alere LFA detects lipoarabinomannan (LAM), a mycobacterial cell surface glycolipid that is shed from metabolically active or degenerating bacterial cells (Wood et al., 2012; Lawn, 2012). In addition to the Alere LFA, LAM has also been the target of several other immunological tests including enzyme linked immunosorbent assays (ELISAs) in both plate and dipstick formats (Dheda et al., 2010; Sarkar et al., 2012). Further, the diagnostic potential of LAM has led it to become the current gold standard biomarker for POC TB diagnostic tests. LAM is shed from the surface of the Mycobacterium into the bloodstream and, after filtration by the kidneys, secreted into urine where it can be detected (Paris et al., 2017). The concentration of LAM in urine of TB-infected, HIV-negative individuals varies from approximately 10 to 1000 pg/mL (Sigal et al., 2018). Higher LAM concentrations are reported in HIV-positive individuals, up to 100 ng/mL, due to a higher overall bacillary load (Sigal et al., 2018). The rapid adoption of biomarker-based LAM testing in HIV endemic areas is expected to help ensure early diagnosis and overcome challenges with diagnosing TB in people with low CD4 counts.

However, implementation of these tests has been limited largely due to their variable sensitivity and specificity. Results from clinical studies and a meta-analysis present pooled sensitivities between 34% and 60% depending on the reference standard (Minion et al., 2011). Increased sensitivity was correlated to the severity of patient immune suppression as the concentration of LAM in urine is thought to depend on the patient's immunocompetence, reflecting increased bacillary load, dissemination of M. tb, and potentially also renal involvement in TB disease in such patients (Minion et al., 2011). Similarly, although most studies have reported high positive predictive values, there remains unexplained variability in specificity, with individual studies reporting values ranging from 79% to 100% (Minion et al., 2011). While poor specificity may reflect the shortcomings of individual assays, it is also possible that false-positive results are due to cross-reactivity with LAM molecules expressed by non-tuberculous mycobacteria (NTM), or related bacteria of the Actinomycetales order (Lawn, 2012; Sarkar et al., 2014; Boehme et al., 2005).

The LAM antigen has three structural domains: (1) a mannosyl-phosphatidyl-myo-inositol linker, (2) a polysaccharide backbone composed of a D-mannan core and D-arabinan branches, and (3) a capping motif on the terminal ends of the D-arabinan branches (Mishra et al., 2011). The linker and backbone are conserved in all LAM variants, while the capping motif differs depending on the species of Mycobacterium (Mishra et al., 2011). There are three classifications of LAM based on variations in their capping motif: (1) ManLAM is capped with α-(1,2)-linked di-mannosyl residues (Manα-(1,2)-Man), (2) PILAM is capped with phospho-myo-inositol, and (3) AraLAM is devoid of capping motifs (Mishra et al., 2011; Nigou et al., 2003). Throughout this manuscript, the specific variant of LAM (ManLAM, PILAM, or AraLAM) will be used where applicable and LAM will be used generally to discuss conserved features, shared properties, and non-specific interactions. ManLAM is unique to the surface envelope of slow-growing, pathogenic mycobacteria, such as M. tb, M. bovis and M. leprae. M. tb and M. bovis ManLAM express the highest number of di-mannosyl capping residues (seven to nine per ManLAM molecule), whereas M. leprae ManLAM averages only one di-mannosyl cap per ManLAM molecule (Nigou et al., 2000; Torrelles et al., 2004). This makes the high abundance of di-mannosyl caps on M. tb ManLAM an especially attractive target for new capture and detection agents (Chan et al., 2015). However, current commercially available LAM detection tests use anti-LAM antibodies that are incapable of distinguishing between the variants of LAM, leading to cross-reactivity and high false-positive detection rates (Kaur et al., 2002). Recently, new antibodies have been developed that are specific for ManLAM, underscoring the need for ManLAM-specific capture agents to improve the specificity of M. tb diagnostic assays (Minion et al., 2011; Chan et al., 2015; Broger et al., 2019; Kawasaki et al., 2019).

### SUMMARY

Thus, in accordance with the present disclosure, a method of diagnosing a Mycobacterium tuberculosis infection in a subject comprising (a) providing a biological sample from a subject; (b) contacting said biological sample with microvirin-N (MVN); and (c) detecting binding of microvirin-N to mannose-capped lipoarabinomannan (ManLAM) in said biological sample, wherein binding of MVN to mannose-capped lipoarabinomannan (ManLAM) in said biological sample indicates that said subject is infected with Mycobacterium tuberculosis. The biological sample may be a fluid sample, such as blood, sputum, or urine. The MVN may be bound to a support, such as a bead, such as a magnetic bead, a microtiter well, a dipstick, a filter, or a membrane. The ManLAM maybe detected when bound to said support and MVN with a lipoarabinomannan (LAM)-binding agent. The LAM binding agent may be a monoclonal antibody (mAb), such as a mAb binding to a D-mannan core structure or a D-arabinan branch structure, such as a tetra-arabinoside motif or hexa-arabinan motif. The LAM-binding agent may comprise a detectable label.

The subject may be suspected as having a Mycobacterium tuberculosis infection or has been exposed to a patient with a Mycobacterium tuberculosis infection. The subject may be at increased risk of contracting a Mycobacterium tuberculosis infection as compared to populational average. The method may further comprise performing a positive control reaction for binding of MVN to ManLAM and/or further comprising performing a negative control reaction for binding of MVN to ManLAM. The method may further comprise performing steps (a)-(c) a second time on said sample or in a different sample from said subject. The limit of detection of ManLAM may be about 500-1500 pg/ml, about 625-1250 pg/ml, or about 1400 pg/ml.

Also provided is an isolated complex comprising microvirin-N bound to (a) a support and (b) mannose-capped lipoarabinomannan (ManLAM). The isolated complex may further be bound to an antibody that is selective for binding to lipoarabinomannan (LAM), such as a labeled anti-LAM antibody, such as wherein the anti-LAM antibody is a mAb binding to a D-mannan core structure or a D-arabinan branch structure, such as a tetra-arabinoside motif or hexa-arabinan motif. The support may be a bead, such as a magnetic bead, a microtiter well, a dipstick, a filter, or a membrane.

In another embodiment, there is provided a kit comprising microvirin-N bound to a support. The kit may further comprise an antibody that is selective for binding to lipoarabinomannan (LAM), such as a labeled anti-LAM antibody, such as wherein the anti-LAM antibody is a mAb binding to a D-mannan core structure or a D-arabinan branch structure, such as a tetra-arabinoside motif or hexa-arabinan motif. The support may be a bead, such as a magnetic bead, a microtiter well, a dipstick, a filter, or a membrane. The kit may further comprise one or more of (i) instructions for performing an assay for detecting mannose-capped lipoarabinomannan in a sample, (ii) mannose-capped lipoarabinomannan, (iii) a reagent comprising a D-mannan core structure or a D-arabinan branch structure, such as a tetra-arabinoside motif or hexa-arabinan motif, or (iv) one or more solutions for diluting a reagent in said kit.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. MVN expression workflow. (1) Transfection of E. coli BL21-DE3 competent cells with pET15b vector (ampicillin resistance gene) expressing microvirin lectin with an N-terminal hexa-histidine tag (MVN-His₆); (2) Inoculation of 1 L Terrific Broth culture medium with overnight starter culture of MVN-His₆-expressing E. coli BS21-DE3 cells followed by incubation of the bulk culture with ampicillin (100 μg/mL) at 37° C., 22 rpm until OD₆₀₀ ˜0.6-0.8 was reached; (3) Cooling of the bulk culture to 18° C., addition of IPTG (1 mM, final concentration), and further overnight incubation (16-18 hrs) of the culture at 18° C., 22 rpm; (4) Harvesting of the MVN-His₆-expressing bacteria by centrifugation; (5) Resuspension of the bacterial pellet in lysis buffer, and lysis of bacteria by passage through an Emulsiflex-C3 High Pressure Homogenizer (Avestin) maintaining lysis pressure at 15,000 psi (three cycles), followed by clarification of the lysate by centrifugation and passage through 0.22 μm syringe filter; (6) Purification of MVN-His₆ by FPLC using a 5 mL HisTrap HP column and elution of fractions with a linear imidazole gradient (0-500 mM); (7) Fractions containing purified MVN-His₆ were pooled and dialyzed against PBS and either used fresh or stored frozen at −80° C.

FIG. 2. Polyacrylamide gel of IMAC fractions. NuPAGE 4-12% gradient gels of the FPLC purification of MVN-His₆ from E. coli BL21-DE3 lysate depicting (i) molecular weight marker (gels 1 & 2, lane 1), (ii) lysate of MVN-His₆-expressing E. coli BL21-DE3 cells (gel 1, lane 2), (ii) Flow-through from HisTrap HP column (gel 1, lane 3), and (iii) IMAC column fractions (gel 1, lanes 4-15 and continued onto gel 2, lanes 2-15). MVN-His₆ appears as a prominent band at a molecular weight of ˜15 kDa in the cell lysate (gel 1, lane 2), but is largely absent in the flow-through (gel 1, lane 3), which indicates the successful binding of MVN-His₆ to the Ni-NTA HisTrap column. Elution of MVN-His₆ under the linear imidazole gradient peaked around the 10^{th} fraction (gel 1, lane 13) and subsequently tapered off as observed by changing intensities of the dark bands at a molecular weight of ˜15 kDa. The elution fractions were pooled as follows for further analysis; Pool 1: gel 1, lanes 6-9; Pool 2: gel 1, lanes 10-13; Pool 3: gel 1, lanes 14-15 and gel 2, lanes 2-6; Pool 4: gel 2, lanes 7-10. 10 μL aliquots of each 2 mL fraction was loaded per well.

FIGS. 3A-B. Polyacrylamide gel of pooled fractions. NuPAGE 4-12% gradient gel of pooled FPLC elution fractions. (FIG. 3A) Pools 2 and 3 were diluted ten-fold prior to being loaded onto the gel. (FIG. 3B) Pools 1 and 4 were loaded undiluted onto the gel. All pools were loaded in three lanes with 2.5, 5, and 10 μL sample, left to right. BSA standards (100, 250, 500, 750, and 1000 ng) were also loaded onto the gel to enable densitometric quantification of MVN-His₆ in the various elution fractions.

FIG. 4. OB-ELISA workflow. 100 μL of ManLAM or PILAM in pooled human urine is added to a clear flat-bottom 96-well plate. A 100 μL aliquot of 2 μg/mL Ab28 conjugated to HRP in 0.5% BSA PBST is then added to the wells. Lastly, 4 μL MVN-functionalized magnetic Dynabeads is added to each well and the plate is incubated on a shaker for 30 minutes. The beads are magnetically separated from the supernatant using a 96-well magnetic separation rack and washed three times with 200 μL PBST. A 100 μL aliquot of TMB One is then added to each well and the plate is incubated on a shaker for 10 minutes while protected from light. Lastly, 100 μL 2 M H₂SO₄ is added to each well to stop the reaction and the signal was measured by absorbance at 450 nm.

FIGS. 5A-D. MVN crystal structure, stability studies, and binding pair assessment. (FIG. 5A) PyMOL rendering of the resolved crystal structure for MVN with the major components labeled.³¹ (FIG. 5B) MVN stability study where binding experiments were performed for 100+ days on MVN stored at 4° C. (FIG. 5C) MVN stability study performed for 2 weeks on MVN stored at RT. (FIG. 5D) FIND Ab28 forms an orthogonal binding pair with MVN.

FIGS. 6A-C. OB-ELISA development. (FIG. 6A) Signal-to-noise ratios for sequential addition vs all-in-one addition format for OB-ELISA showing no significant different (p-value=0.21). (FIG. 6B) Optimization of detection antibody concentration. (FIG. 6C) Optimization of bead number and type where Bead 1 was functionalized with 60 μg MVN per 100 μL beads and Bead 2 was functionalized with 30 μg MVN per 100 μL beads.

FIGS. 7A-B. Results of the MVN-based on-bead ELISA. (FIG. 7A) Standard curves of ManLAM and PILAM spiked into pooled human urine. (FIG. 7B) Using larger urine sample volumes in the OB-ELISA allows the resulting signal to be increased proportionally.

FIGS. 8A-D. Antibody-based ELISA. (FIG. 8A) The concentration of capture antibody Ab170 was optimized and 4 μg/mL was chosen. (FIG. 8B) The percentage of BSA in the blocking buffer was optimized and 1.25% was chosen. (FIG. 8C) The concentration of detection antibody Ab28 conjugated to HRP was optimized and 0.5 μg/mL was chosen. (FIG. 8D) Standard curves of ManLAM and PILAM spiked into urine were used to determine the LODs of 729 pg/mL and 360 pg/mL, respectively.

FIG. 9. ManLAM and PILAM titration curves on Alere LFA. Titration curves of M. tb ManLAM or M. smegmatis PILAM on the Alere LFA. The visual LODs were found to be 1.25 ng/mL ManLAM and 0.625 ng/mL PILAM.

FIGS. 10A-B. (FIG. 10A) 2-fold, 7-fold, and 14-fold concentration factors were compared for the highest signal-to-noise. (FIG. 10B) The LOD of the OB-ELISA on 14-fold concentrated mock urine samples was determined to be 274 pg/mL.

FIG. 11. Normalized absorbance values for the 58 clinical urine samples.

FIGS. 12A-B. Box-and-whisker plots showing the median and interquartile range for different populations: (FIG. 12A) HIV status and (FIG. 12B) country of origin.

FIGS. 13A-B. (FIG. 13A) Box-and-whisker plots by TB-status and sex showing no association. (FIG. 13B) Scatter plot showing no association between absorbance and age for TB-positive or TB-negative samples.

FIGS. 14A-B. (FIG. 14A) Receiver operating characteristic (ROC) curves for the clinical urine samples, overall and in sub-populations by HIV-status and country of origin. (FIG. 14B) The area under the curve was determined for each ROC curve.

### DESCRIPTION OF ILLUSTRATIVE EMBODIMENTS

As discussed above, Mycobacterium tuberculosis continues to present a major health challenge that, while being very deadly, can be effectively treated with early diagnosis. In this study, the inventors expressed microvirin-N (MVN), a 14.3 kDa cyanobacterial lectin that specifically binds the Manα-(1,2)-Man di-mannosyl caps of ManLAM with sub-picomolar binding affinity and demonstrate its utility in ManLAM TB diagnostic tests (Kehr et al., 2006; Huskens et al., 2010; Shahzad-ul-Hussan et al., 2011). They characterized the binding of MVN to ManLAM using bio-layer interferometry and have shown that MVN does not bind to non-mannose-capped lipoarabinomannan, such as lipoarabinomannan capped with a phosphate-inositol group (PILAM) found on the related bacteria Mycobacterium smegmatis. A “sandwich” assay pairing of MVN with a monoclonal antibody that binds an orthogonal linear hexa-arabinan (Ara₆) motif located in the branches of LAM was developed and employed as highly specific, sensitive, and stable diagnostic test for M. tb ManLAM. To the best of the inventors' knowledge, this is the first study to demonstrate the use of a lectin-based molecular recognition element to selectively capture and detect ManLAM as a tuberculosis diagnostic test

These and other aspects of the disclosure are described in detail below.

### I. MYCOBACTERIUM TUBERCULOSIS

Mycobacterium tuberculosis (M. tb) is a species of pathogenic bacteria in the family Mycobacteriaceae and the causative agent of tuberculosis. First discovered, M. tuberculosis has an unusual, waxy coating on its cell surface primarily due to the presence of mycolic acid. This coating makes the cells impervious to Gram staining, and as a result, M. tuberculosis can appear either Gram-negative or Gram-positive. Acid-fast stains such as Ziehl-Neelsen, or fluorescent stains such as auramine are used instead to identify M. tuberculosis with a microscope. The physiology of M. tuberculosis is highly aerobic and requires high levels of oxygen. Primarily a pathogen of the mammalian respiratory system, it infects the lungs. The most frequently used diagnostic methods for tuberculosis are the tuberculin skin test, acid-fast stain, culture, and polymerase chain reaction.

M. tuberculosis is part of a complex that has at least 9 members: M. tuberculosis sensu stricto, M. africanum, M. canetti, M. bovis, M. caprae, M. microti, M. pinnipedii, M. mungi, and M. orygis. It requires oxygen to grow, does not produce spores, and is nonmotile. M. tuberculosis divides every 18-24 hours. This is extremely slow compared with other bacteria, which tend to have division times measured in minutes (Escherichia coli can divide roughly every 20 minutes). It is a small Bacillus that can withstand weak disinfectants and can survive in a dry state for weeks. Its unusual cell wall is rich in lipids such as mycolic acid and is likely responsible for its resistance to desiccation and is a key virulence factor.

Humans are the only known reservoirs of M. tuberculosis. A misconception is that M. tuberculosis can be spread by shaking hands, making contact with toilet seats, sharing food or drink, sharing toothbrushes, or kissing. It can only be spread through air droplets originating from a person who has the disease either coughing, sneezing, speaking, or singing.

When in the lungs, M. tuberculosis is phagocytosed by alveolar macrophages, but they are unable to kill and digest the bacterium. Its cell wall prevents the fusion of the phagosome with the lysosome, which contains a host of antibacterial factors. Specifically, M. tuberculosis blocks the bridging molecule, early endosomal autoantigen 1 (EEA1); however, this blockade does not prevent fusion of vesicles filled with nutrients. Consequently, the bacteria multiply unchecked within the macrophage. The bacteria also carry the UreC gene, which prevents acidification of the phagosome. In addition, production of the diterpene isotuberculosinol prevents maturation of the phagosome. The bacteria also evade macrophage-killing by neutralizing reactive nitrogen intermediates. More recently, it has been shown that M. tuberculosis secretes and covers itself in 1-tuberculosinyladenosine (1-TbAd), a special nucleoside that acts as an antacid, allowing it to neutralize pH and induce swelling in lysosomes. 1-TbAd is encoded by the gene Rv3378c.

It was also recently demonstrated that in M. tuberculosis infections, PPM1A levels were upregulated, and this in turn would impact the normal apoptotic response of macrophages to clear pathogens, as PPM1A is involved in the intrinsic and extrinsic apoptotic pathways. Hence, when PPM1A levels were increased, the expression of it would inhibit the two apoptotic pathways. With kinome analysis, the JNK/AP-1 signaling pathway was found to be a downstream effector that PPM1A has a part to play in, and the apoptotic pathway in macrophages are controlled in this manner. As a result of having apoptosis being suppressed, it provides M. tuberculosis with a safe replicative niche, and so the bacteria is able to maintain a latent state for a prolonged period of time.

Protective granulomas are formed due to the production of cytokines and upregulation of proteins involved in recruitment. Granulotomatous lesions are important in both regulating the immune response and minimizing tissue damage. Moreover, T cells help maintain Mycobacterium within the granulomas.

The ability to construct M. tuberculosis mutants and test individual gene products for specific functions has significantly advanced the understanding of its pathogenesis and virulence factors. Many secreted and exported proteins are known to be important in pathogenesis. For example, one such virulence factor is cord factor (trehalose dimycolate), which serves to increase survival within its host. Resistant strains of M. tuberculosis have developed resistance to more than one TB drug, due to mutations in their genes. In addition, pre-existing first-line TB drugs such as rifampicin and streptomycin have decreased efficiency in clearing intracellular M. tuberculosis due not being able to effectively penetrate the macrophage niche.

JNK plays a key role in the control of apoptotic pathways—intrinsic and extrinsic. In addition, it is also found to be a substrate of PPM1A activity, hence the phosphorylation of JNK would cause apoptosis to occur. Since PPM1A levels are elevated during M. tuberculosis infections, by inhibiting the PPM1A signaling pathways, it could potentially be a therapeutic method to kill M. tuberculosis infected macrophages by restoring its normal apoptotic function in defense of pathogens. Therefore, by targeting the PPM1A-JNK signaling axis pathway, it could eliminate M. tuberculosis infected macrophages.

The ability to restore macrophage apoptosis to M. tuberculosis infected ones could improve the current tuberculosis chemotherapy treatment, as TB drugs can gain better access to the bacteria in the niche. Therefore, decreasing the treatment time periods for M. tuberculosis infections.

Symptoms of M. tuberculosis include coughing that lasts for more than three weeks, hemoptysis, chest pain when breathing or coughing, weight loss, fatigue, fever, night sweats, chills, and loss of appetite. M. tuberculosis also has the potential of spreading to other parts of the body. This can cause blood in urine if the kidneys are affected, and back pain if the spine is affected.

The genome of the H37Rv strain was published in 1998. Its size is 4 million base pairs, with 3,959 genes; 40% of these genes have had their function characterized, with possible function postulated for another 44%. Within the genome are also six pseudogenes.

The M. tuberculosis genome was sequenced in 1998 and contains 250 genes involved in fatty acid metabolism, with 39 of these involved in the polyketide metabolism generating the waxy coat. Such large numbers of conserved genes show the evolutionary importance of the waxy coat to pathogen survival. Furthermore, experimental studies have since validated the importance of a lipid metabolism for M. tuberculosis, consisting entirely of host-derived lipids such as fats and cholesterol. Bacteria isolated from the lungs of infected mice were shown to preferentially use fatty acids over carbohydrate substrates. M. tuberculosis can also grow on the lipid cholesterol as a sole source of carbon, and genes involved in the cholesterol use pathway(s) have been validated as important during various stages of the infection lifecycle of M. tuberculosis, especially during the chronic phase of infection when other nutrients are likely not available.

About 10% of the coding capacity is taken up by the PE/PPE gene families that encode acidic, glycine-rich proteins. These proteins have a conserved N-terminal motif, deletion of which impairs growth in macrophages and granulomas. Nine noncoding sRNAs have been characterized in M. tuberculosis, with a further 56 predicted in a bioinformatics screen.

In 2013, a study on the genome of several sensitive, ultraresistant, and multiresistant M. tuberculosis strains was made to study antibiotic resistance mechanisms. Results reveal new relationships and drug resistance genes not previously associated and suggest some genes and intergenic regions associated with drug resistance may be involved in the resistance to more than one drug. Noteworthy is the role of the intergenic regions in the development of this resistance, and most of the genes proposed in this study to be responsible for drug resistance have an essential role in the development of M. tuberculosis.

M. tuberculosis is a clonal organism and does not exchange DNA via horizontal gene transfer. This, possibly combined with a relatively low rate of evolution, might explain why the evolution of resistance has been relatively slow in the species compared to some other major bacterial pathogens. However, ABR is a very serious and growing challenge. Worst hit are countries in the former Soviet republics, where ABR evolved and spread at explosive levels following the fall of the Soviet Union. An extreme example is Belarus, where a third of all new cases of tuberculosis are multidrug-resistant. Multidrug-resistant tuberculosis requires prolonged treatment with expensive and often toxic drugs, and treatment failure is common.

Multidrug-resistant Tuberculosis (MDR-TB) is caused by an organism that is resistant to at least isoniazid and rifampin, the two most powerful TB drugs. These medications are utilized to treat all people with TB illness. The majority of people with TB are cured by a strictly followed, half-year medicate routine that is provided to patients with support and supervision. Inappropriate or incorrect use of antimicrobial medications, or utilization of ineffectual plans of medications and untimely treatment interruption can cause drug resistance, which can then be transmitted, especially in crowded settings such as prisons and hospitals. In 2016, an estimated 490,000 people worldwide developed MDR-TB, and an additional 110,000 people with rifampicin-resistant TB were also newly eligible for MDR-TB treatment. The countries with the largest numbers of MDR-TB cases (47% of the global total) were China, India and the Russian Federation.

### II. MICROVIRIN-N (MVN)

Microvirin-N (MVN) is a 14.3 kDa cyanobacterial lectin, a carbohydrate-binding protein, that specifically binds to terminal Manα(1-2)Manα moieties. The discovery of MVN was reported in Kehr et al. (2006) and its binding properties were investigated by Huskens et al. (2010). Since then, it has been investigated for anti-HIV-1 activity and as a neutralizing agent for hepatitis C viral infections. The protein and mRNA accession numbers for MVN are 2Y1S (Protein Data Bank) and AM041066 (European Nucleotide Archive).

MVN is a 108aa protein encoded in the Microcystis genome and is composed of two similar domains. An amino acid alignment of the individual domains of MVN and cyanovirin-N (CV-N) show an overall low similarity although four conserved cysteine residues have been identified. These cysteine residues are involved in internal disulphide bridge formation that is crucial for the structure and activity of CV-N (Gustafson et al., 1997). MVN contains two additional cysteine residues in its C-terminal domain. Amino acid residues in CV-N that form part of the primary carbohydrate binding site are present or conservatively exchanged in MVN (N42, N53, E56, T57) while others are absent. Residues involved in the secondary carbohydrate binding site are partly conserved. Sequences with similarities to MVN were also found to be encoded in some fungal genomes, such as Gibberella zeae PH-1, Neurospora crassa, Tuber borchii, Aspergillus nidulans FGSC A4 and Magnaporthe grisea 70-15.

The apparent Mᵣ of is 24 kDa and is consistent with a MVN dimer. Under non-reducing conditions, disulphide bonds are present that prevent dimerization of the protein. The total mass of a His₆-tagged MVN was determined by NSI FTICR MS under non-reducing conditions and the measured value of m/z 14215.414 was in accordance with the simulated value for a His-MVN monomer without N-terminal methionine. It also indicated the presence of two internal disulphide bonds.

### III. DETECTION METHODS

A. Assay Formats

In still further embodiments, the present disclosure concerns methods for binding, quantifying and otherwise generally detecting ManLAM, and in particular in a sample from a subject at risk of, suspected of having or exposed to tuberculosis. A wide variety of assay formats are contemplated, but specifically those that would be used to detect ManLAM in a fluid obtained from a subject, such as saliva, blood, plasma, sputum, semen or urine. The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some detection methods include methods analogous to an enzyme linked immunosorbent assay (ELISA), radioimmunoassay (RIA), immunoradiometric assay, fluoroimmunoassay, chemiluminescent assay, bioluminescent assay, FAC analysis, and Western blot to mention a few. The steps of various analogous immunodetection methods have been described in the scientific literature, such as, e.g., Doolittle and Ben-Zeev (1999), Gulbis and Galand (1993), De Jager et al. (1993), and Nakamura et al. (1987). In general, the methods employed here include obtaining a sample suspected of containing ManLAM/M. tuberculosis and contacting the sample with MVN or a ManLAM binding fragment thereof under conditions effective to allow the formation of complexes.

MVN may be linked to a solid support, such as in the form of a column matrix or microtiter well, and the sample suspected of containing ManLAM/M. tuberculosis will be applied to the immobilized MVN. The unwanted components will be washed from the column, leaving the ManLAM/M. tuberculosis complexed to the MVN, which is then collected by removing the ManLAM/M. tuberculosis from the column.

MVN may also be linked to a magnetic bead as an alternative solid support. The MVN-functionalized magnetic beads will be added to the sample suspected of containing ManLAM/M. tuberculosis. MVN will capture ManLAM from the solution, resulting in the MVN-ManLAM complex immobilized to the magnetic bead. The beads can be held in place with a magnet while unwanted components are removed. The complex can then be detected on the bead (as described further in the Materials and Methods section below) or released from the bead to detect in a downstream assay.

The methods also include methods for detecting and quantifying the amount of ManLAM/M. tuberculosis or related components in a sample and the detection and quantification of any MVN complexes formed during the binding process. Here, one would obtain a sample suspected of containing ManLAM/M. tuberculosis and contact the sample with MVN or a ManLAM binding fragment thereof, followed by detecting and quantifying the amount of MVN complexes formed under the specific conditions. In terms of detection, the biological sample analyzed may be any sample that is suspected of containing ManLAM/M. tuberculosis, such as a tissue section or specimen, a homogenized tissue extract, a biological fluid, including blood, sputum and serum, or a secretion, such as feces or urine.

Contacting the chosen biological sample with MVN under effective conditions and for a period of time sufficient to allow the formation of complexes (primary complexes) is generally a matter of simply adding the MVN composition to the sample and incubating the mixture for a period of time long enough for the MVN to form complexes with, i.e., to bind to ManLAM/M. tuberculosis. After this time, the sample-MVN composition be washed to remove any non-specifically bound species, allowing only ManLAM/M. tuberculosis specifically bound within the primary complexes to be detected.

In general, the detection of complex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as one that binds to LAM/M. tuberculosis, as is known in the art.

The second binding ligand employed in the detection may itself be linked to a detectable label, wherein one would then simply detect this label, thereby allowing the amount of the primary immune complexes in the composition to be determined. The second binding ligand is often an antibody. The primary complexes are contacted with the labeled, secondary binding ligand or antibody under effective conditions and for a period of time sufficient to allow the formation of secondary complexes. The secondary complexes are then generally washed to remove any non-specifically bound labeled LAM binding antibodies or ligands, and the remaining label in the secondary complexes is then detected.

Further methods include the detection of primary complexes by a two-step approach. A second binding ligand, such as an antibody that has binding affinity for LAM/M. tuberculosis, is used to form secondary complexes, as described above. After washing, the secondary complexes are contacted with a third binding ligand or antibody that has binding affinity for the second binding ligand, again under effective conditions and for a period of time sufficient to allow the formation of complexes (tertiary complexes). The third ligand or antibody is linked to a detectable label, allowing detection of the tertiary complexes thus formed. This system may provide for signal amplification if this is desired.

Another known method of detection is that analogous to immuno-PCR (Polymerase Chain Reaction) methodologies. The PCR method is similar to the Cantor method up to the incubation with biotinylated DNA; however, instead of using multiple rounds of streptavidin and biotinylated DNA incubation, the DNA/biotin/streptavidin/MVN complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single ManLAM molecule.

Binding assays for ManLAM may be performed in a fashion analogous to enzyme linked immunosorbent assays (ELISAs), radioimmunoassays (RIA), Western blotting, dot blotting, and FACS analyses.

“Under conditions effective to allow complex (MVN/ManLAM) formation” means that the conditions preferably include diluting the sample with solutions such including BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation, a contacted surface generally is washed so as to remove non-complexed material. A preferred washing procedure includes washing with a solution such as PBS/Tween, or borate buffer. Following the formation of specific complexes between the test sample and the originally bound material, and subsequent washing, the occurrence of even minute amounts of complexes may be determined.

Colorimetric detection often employs an enzyme that will generate color development upon incubating with an appropriate chromogenic substrate. Thus, for example, one will desire to contact or incubate the second complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween). After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzothiazoline-6-sulfonic acid (ABTS), or H₂O₂, in the case of peroxidase as the enzyme label. Quantification is then achieved by measuring the degree of color generated, e.g., using a visible spectra spectrophotometer.

Another particularly useful assay format is the lateral flow assay, also known as lateral flow chromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically, there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

MVN may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

B. Kits

In still further embodiments, the present disclosure concerns kits for use with the detection methods described above. As MVN may be used to detect ManLAM/M. tuberculosis, MVN and ManLAM/M. tuberculosis standards may be included in the kit. The kits may thus comprise, in suitable container means, MVN and a detection reagent for LAM/M. tuberculosis.

In certain embodiments, the MVN may be pre-bound to a solid support, such as a column matrix, filter, magnetic bead, and/or well of a microtiter plate. The detection reagents of the kit may take any one of a variety of forms, including those with detectable labels that are associated with or linked to the detection reagent. Detectable labels that are associated with or attached to a secondary binding ligand are also contemplated. Exemplary secondary ligands are those secondary antibodies that have binding affinity for LAM/M. tuberculosis.

The kits may further comprise a suitably aliquoted composition of the ManLAM/M. tuberculosis, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the MVN may be placed, or preferably, suitably aliquoted. The kits of the present disclosure will also typically include a means for containing the MVN and any other reagent containers in close confinement for commercial sale. Such containers may include injection or blow-molded plastic containers into which the desired vials are retained.

### IV. ANTIBODY CONJUGATES

As discussed above, the present disclosure may employ an antibody linked to at least one agent to form an antibody conjugate permitting detection of the MVN+ManLAM/M. tuberculosis complex. In order to increase the efficacy of antibody molecules as diagnostic or therapeutic agents, it is conventional to link or covalently bind or complex at least one desired molecule or moiety. Such a molecule or moiety may be, but is not limited to, at least one reporter molecule. A reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles, ligands, such as biotin or nucleic acid tags.

Antibody conjugates are generally preferred for use as diagnostic agents. Many appropriate detection agents are known in the art, as are methods for their attachment to antibodies (see, for e.g., U.S. Pat. Nos. 5,021,236, 4,938,948, and 4,472,509). The imaging moieties used can be enzymes, paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

### V. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Expression and purification of MVN. Competent E. coli BL21-DE3 bacteria were transformed with the MVN-pET15b plasmid (190 ng plasmid per 100 μL competent bacteria) by heat shock. The plasmid-transformed bacteria were plated onto LB Agar plates containing ampicillin (100 μg/mL). Colonies were selected from the plates and stored as a glycerol stock. A 50 mL aliquot of LB medium containing 100 μg/mL ampicillin was inoculated with MVN-pET15b transformed BL21-DE3 bacteria as a starter culture, and allowed to grow at 37° C., 22 rpm overnight (˜16-18 hours). 1 L sterilized TB Broth was inoculated with overnight starter culture until the OD₆₀₀ reading reached ˜0.1. Ampicillin was then added to a final concentration of 100 μg/mL. The culture was allowed to grow at 37° C., 22 rpm until the OD₆₀₀ reading reached ˜0.6-0.8. The culture was cooled to 18° C. and isopropyl β-D-1-thiogalactopyranoside (IPTG) was added to final concentration of 1 mM. Cultures were further incubated overnight (˜16-18 hours) at 18° C., 22 rpm. Bacterial cells were harvested via centrifugation and either used fresh or stored frozen (FIG. 1).

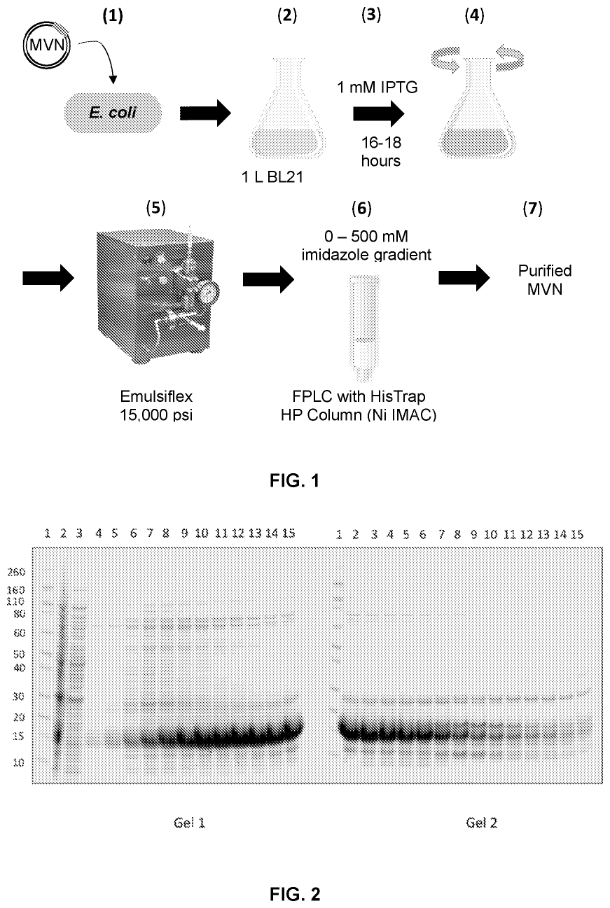

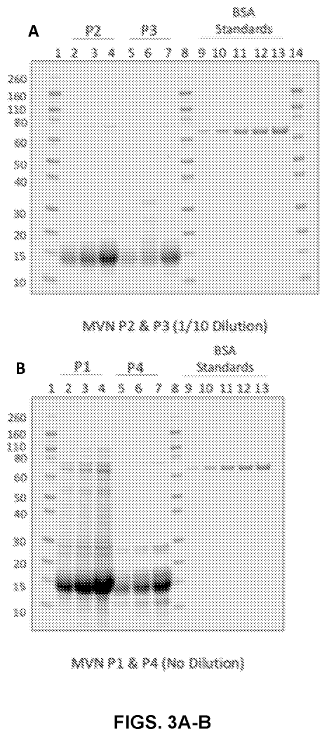

Cell pellets were resuspended in 150 mL phosphate buffer (25 mM NaH₂PO₄, 500 mM NaCl, 10% glycerol, pH 8) containing 2 μg/mL lysozyme (Sigma: Cat. No. L6876), 1 mL protease cocktail (Sigma: Cat. No. P8849), and powdered DNase I (Sigma: Cat. No. DN25). The cell suspension was then passed through an Emulsiflex-C3 High Pressure Homogenizer (Avestin) three times maintaining lysis pressure at 15,000 psi. The lysate was centrifuged and the resulting supernatant was clarified by filtering through a 0.22 μm filter. The clarified lysate was loaded onto a 5 mL HisTrap HP column (GE: 17-5248-02) and purified by FPLC. MVN was eluted with a linear imidazole gradient from 0-500 mM (FIG. 2). Fractions containing MVN were pooled and dialyzed against PBS overnight at 4° C. Protein concentration was determined by quantification gel—Invitrogen NuPAGE 4-12% gradient gel run in 1×MES buffer with densitometry performed using the Image Studio Lite Version 5.2 software (FIGS. 3A-B).

Bioconjugation. MVN (1.36 mg/mL) was conjugated to biotin using a 20× molar equivalence of EZ-Link NHS-PEG₄-Biotin (Thermo Scientific: Cat. No. 21329) in DI water. Following a 30-minute incubation step at room temperature, the MVN-biotin conjugate was purified from excess biotin reagent using a 7 k MWCO Zeba desalting column (Thermo Scientific: Cat. No. 89883). The concentration of the resulting MVN-biotin conjugate was determined by micro-volume analysis using a BioTek Take3 plate on a BioTek Synergy H4 plate reader with the lysozyme setting.

Anti-LAM antibodies Ab25, Ab28, and Ab170 were procured from the Foundation for Innovative New Diagnostics (FIND). FIND Ab25, Ab28, and Ab170 were conjugated to biotin with a 20× molar equivalence of EZ-Link NHS-PEG₄-Biotin (Thermo Scientific: Cat. No. 21329) in DI water. Following a 30-minute incubation step at room temperature, the antibody-biotin conjugates were purified from excess biotin reagent using 7 k MWCO Zeba desalting columns (Thermo Scientific: Cat. No. 89883). The concentration of the resulting antibody-biotin conjugates was determined by micro-volume analysis using a BioTek Take3 plate with the IgG setting.

FIND Ab28 was conjugated to horseradish peroxidase (HRP) using EZ-Link Plus Activated Peroxidase (Thermo Scientific: Cat. No. 31489). The single-use tube was dissolved in DI H₂O and added to the antibody, followed by sodium cyanoborohydride. The reaction was incubated for 1 hour at room temperature. Quenching buffer was added and allowed to react for 15 minutes. The resulting antibody-conjugate was transferred to an Amicon Ultra-0.5 mL Centrifugal Filter Ultracel—100K spin filter (Millipore Sigma: Cat. No. UFC510024) and centrifuged for 10 minutes at 14,000×g. The spin filters were washed three times with PBS then inverted into new collection tubes and centrifuged for 2 minutes at 1,000×g. The concentration of the antibody-conjugate was determined by microvolume analysis using a BioTek Take3 plate with the IgG setting.

Binding characterization of molecular recognition elements by bio-layer interferometry. Anti-LAM antibodies CS35 and CS40 were procured from the Biodefense and Emerging Infections Research Resources Repository (BEI Resources). MVN, Ab25, Ab28, and Ab170 were conjugated to biotin as described above. Following the conjugation, the molecular recognition elements were subjected to binding experiments on the ForteBio Octet RED96 to determine binding affinity to M. tb H37Rv ManLAM, M. leprae ManLAM, and M. smegmatis PILAM.¹⁸ Dip and Read Streptavidin biosensors (ForteBio: Cat. No. 18-5019) were used for the binding experiments with biotinylated MVN, Ab25, Ab28, and Ab170 and Dip and Read Anti-Mouse Fc capture biosensors (ForteBio: Cat. No. 18-5088) were used for the binding experiments with CS35 and CS40.

The biosensors were first introduced to a biosensor buffer (0.1% BSA (Fisher: BioReagents: Cat. No. BP9706100), 0.02% Tween 20 in PBS (Fisher BioReagents: Cat. No: BP337100) for a 300 second equilibration step. The biosensors were then transferred to wells containing MVN-biotin (0.125 μg/mL) or antibody-biotin (0.5 μg/mL) for 400 seconds. The loaded biosensors were moved to wells containing biosensor buffer for a 60 second baseline step and then transferred to sample wells containing a two-fold serially diluted range of M. tb H37Rv ManLAM with a buffer reference well for a 400 second association step. The two-fold serial dilution began at an initial concentration of ManLAM such that the starting concentration of ManLAM was at least 10× higher than the expected K_{D} and the lowest concentration was 2× lower than the expected K_{D} (Chan et al., 2015). Lastly, the biosensors were moved back to the baseline buffer wells for a 900 second dissociation step. The same procedure was used with Dip and Read Ni-NTA biosensors (ForteBio: Cat. No. 18-5101) to determine the binding affinity of MVN-His₆, except the biosensor buffer was 0.02% Tween 20 in PBS and MVN was loaded onto the biosensor at 0.5 μg/mL. Inside the ForteBio software, a global 1:1 fit model was selected to derive the kinetic (kₒₙ, k_{off}) and equilibrium (K_{D}) data.

Stability test of MVN by bio-layer interferometry. MVN was stored at either 4° C. or RT. Repeated binding experiments at set time points were performed on MVN to determine changes in its binding affinity toward M. tb H37Rv ManLAM over time as a representation of stability. For the batch stored at 4° C., measurements were made on days 0, 1, 2, 3, 4, 8, 12 (excluded by outlier test), 16, 22, 29, 36, 43, 49 (measured twice using two batches of ManLAM and averaged), 63, 77, 91, and 105). For the batch stored at RT, aliquots were moved from the freezer to RT on days 0, 1, 2, 3, 4, 5, 6, 8, 10, 12, and 14; binding experiments were performed with each aliquot on the last day. The binding experiments were performed in the same manner as before.

Binding pair evaluation by bio-layer interferometry. The binding pair evaluation was run on the Octet RED96, equipped with Dip and Read Ni-NTA (ForteBio: Cat. No. 18-5101). The biosensors were first introduced to a biosensor buffer (0.02% Tween 20 in PBS) for a 300 second equilibration step, followed by a loading step where the biosensors were transferred to wells containing 0.7 μg/mL MVN-His₆ for 400 seconds. The loaded biosensors were transferred to wells containing biosensor buffer for a 60 second baseline step and then transferred to sample wells containing 0.325 μg/mL M. tb H37Rv ManLAM for a 400 second association step. The biosensors were returned to the baseline wells for a 60 second baseline step and then transferred to wells containing 12 μg/mL non-conjugated Ab28 for another 400 second association step. Lastly, the biosensors were returned to the baseline buffer wells for a 900 second dissociation step.

On-bead ELISA (OB-ELISA). First, Dynabeads MyOne™ Streptavidin T1 beads (Invitrogen: Cat. No. 65601) were functionalized with MVN-biotin conjugate. MVN was conjugated to biotin as above. The streptavidin beads were washed three times with PBS using a magnetic separation rack (Invitrogen: Cat. No CS15000). MVN-biotin conjugate was added at a ratio of either 60 μg MVN per 100 μL beads (Bead 1) or 30 μg MVN per 100 μL beads (Bead 2). The resulting mixture was incubated for 30 minutes at room temperature. The beads were then washed three times with PBS and blocked with excess D-biotin in PBS for 30 minutes. Lastly, the beads were washed three times with PBS and re-suspended in PBST (PBS, 0.1% Tween 20).

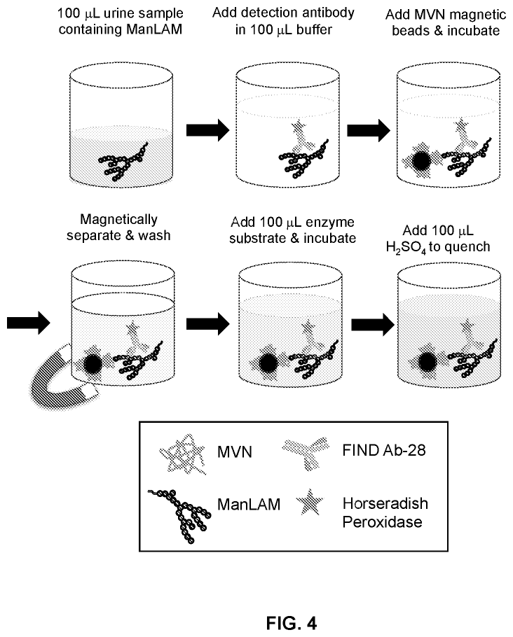

Solutions (100 μL) of ManLAM or PILAM in 0.1% BSA PBST or pooled human urine (Innovative Research: Cat. No. IRHUURE1000ML) were placed in a clear flat-bottom 96-well plate (FIG. 4). A 100 μL aliquot of 2 μg/mL Ab28 conjugated to HRP in 0.5% BSA PBST was then added to the wells. Lastly, 4 μL MVN-functionalized magnetic Dynabeads were added to each well and the plate was incubated on a shaker for 30 minutes. The beads were magnetically separated from the supernatant using a 96-well magnetic separation rack (EdgeBio: Cat. No. 57624) and washed three times with 200 μL PBST. A 100 μL aliquot of TMB One (Promega: Cat. No. G7431) was added to each well containing beads and the plate was incubated on a shaker for 10 minutes while protected from light. Lastly, 100 μL 2 M H₂SO₄ was added to each well to stop the reaction and the signal was measured by absorbance at 450 nm to the thousandths place on a microplate reader. When larger volumes of urine were tested, the entire procedure was scaled accordingly.

The absorbance values were doubled to account for a 1:1 dilution. For both ManLAM and PILAM, absorbance vs concentration was plotted. The average signal of the blank was subtracted to correct for background. The limit of detections was calculated as 3SD_{blank}/slope. Intra-assay variation was determined by calculating the average relative standard deviation of each triplicate measurement on the same plate.

Antibody-based plate ELISA. Solutions (100 μL) of 4 μg/mL Ab170 capture antibody were added to an Immulon 2 HB clear flat-bottomed 96-well plate (ThermoFisher: Cat. No. 3455) and allowed to incubate for 1 hour at room temperature. Wells were washed three times with 235 μL PBST (PBS, 0.1% Tween 20). 235 μL of 1.25% BSA PBST were added to the wells and allowed to incubate for 2 hours, followed by 3 washes with 235 μL PBST. Next, 100 μL of ManLAM or PILAM spiked into urine were added to the wells, and the plate was allowed to incubate for 2 hours, followed by 4 washes with 235 μL PBST. A solution (100 μL) of 0.5 μg/mL detection antibody Ab28 conjugated to HRP (conjugation described above) was added to the wells and allowed to incubate for 1 hour, followed by 5 washes with 235 μL PBST. A 100 μL aliquot of TMB One was added to each well and the plate was incubated on a shaker for 10 minutes while protected from light. Lastly, 100 μL 2 M H₂SO₄ was added to each well to stop the reaction and the signal was measured by absorbance at 450 nm on a microplate reader. For both ManLAM and PILAM, absorbance vs concentration was plotted. The limit of detections was calculated as 3SD_{blank}/slope.

Alere LFAs. Alere LFAs were performed according to the manufacturer's instructions—60 μL of ManLAM or PILAM spiked into urine was applied to the Alere LFAs (Lot No. 170423) in triplicate. After 25 minutes, the LFAs were read on the Qiagen ESEQuant LFR—Lateral Flow Reader to determine the test line area and visible test lines were noted.

### Example 2—Results

New approaches for TB control and care are urgently needed. As accurate diagnosis is one of the cornerstones of TB control, POC diagnostic tests serve as the most practical tests to enable well-timed diagnosis, guide the appropriate treatment, and curb the transmission and unacceptably high number of deaths due to this otherwise curable disease (WHO 2018; Goletti et al., 2016). Conventional direct smear, bacterial culture and nucleic acid amplification tests, such as GeneXpert MTB/RIF, require biohazardous M. tb-infected sputum samples to yield a positive diagnosis of TB (Goletti et al., 2016). Yet, it is difficult for many active TB patients to produce sputum samples, including those co-infected with HIV, diabetes patients, and children (Goletti et al., 2016). Alternatively, urine analysis obviates the biohazards associated with sputum handling and can be easily collected from all patients (Lawn, 2012; Sarkar et al., 2014). LAM is a particularly advantageous biomarker to detect in urine because it is heat stable and does not readily degrade over time (Lawn, 2012). The sensitivity and specificity shortcomings associated with current urine-based LAM tests are the consequence of several parameters including the characteristics of the capture/detection reagents, condition of the urine sample, immune status of the patient, and the variable concentration of excreted LAM that is dependent on the manifestations of co-infected disease (Sarkar et al., 2014). Of these factors, the selection of the test-capture reagents is the only one that researchers can improve upon to enhance the detection rate of TB-positive patients.

Current LAM detection tests employ either polyclonal or monoclonal antibodies. Polyclonal antibodies raised against LAM are more likely to recognize multiple antigenic epitopes compared to monoclonal antibodies but carry the risk of batch-to-batch variation and cross-reactivity (Lipman et al., 2005). Although monoclonal antibodies (mAbs) target a single epitope, the majority of mAbs generated against LAM target the conserved D-arabinan branches and D-mannan polysaccharide backbone of the antigen (Amin et al., 2018). These D-arabinan and D-mannan epitopes are also conserved in LAM-like polymers produced by other actinomycetes. As previously mentioned, M. tb, as well as other slow-growing, pathogenic mycobacteria species, express ManLAM, whereas non-pathogenic mycobacteria, such as M. smegmatis and M. chelonae, express PILAM and AraLAM, respectively (Mishra et al., 2011). Therefore, targeting the mannose caps that make ManLAM distinct would enhance the specificity of the diagnostic test. Recently, antibodies that exclusively target the unique Manα-(1,2)-Man and 5-methylthio-D-xylose (MTX) capping residues of ManLAM have been produced (Sigal et al., 2018; Chan et al., 2015; Broger et al., 2019; Kawasaki et al., 2019). However, the MTX capping residue is present in low abundance on M. tb ManLAM, typically only one MTX residue per ManLAM molecule, and may be a suboptimal moiety to target on M. tb ManLAM for diagnostic purposes (Treumann et al., 2002).

The discovery of MVN, a highly specific Manα-(1,2)-Man binding lectin, was reported by Kehr et al. (2006). Since then, it has been investigated for anti-HIV-1 activity and as a neutralizing agent for hepatitis C viral infections (Huskens et al., 2010; Shahzad-ul-Hussan et al., 2017; Min et al., [DATE?]). To evaluate the feasibility of MVN to selectively capture ManLAM for use in next generation TB diagnostic tests, MVN was subjected to bio-layer interferometry (BLI) experiments with procurable LAM variants. LAM antibodies procured from the BEI repository and FIND were also evaluated employing the same design of experiments. When comparing the quantitative binding data between MVN and commercially available anti-LAM antibodies, MVN exhibited the highest binding affinity with markedly improved specificity as it did not bind to PILAM (Table 1). No appreciable binding was found for either of the anti-LAM polyclonal antibody matrices. This could be caused by the immobilization of non-specific antibodies on the biosensor which would skew the global binding data. This observation further substantiates the argument that polyclonal antibodies are not ideal for employment in quality-controlled diagnostic tests.

The selective capture of ManLAM with MVN affords the ability to discriminate between pathogenic and non-pathogenic mycobacteria, which is crucial to avoid subjecting non-TB infected individuals to long durations of anti-TB therapy that can cause a host of potential side effects. However, in addition to M. tb, other pathogenic mycobacterial species that express ManLAM, such as M. bovis and M. leprae, would also be detected by MVN.¹⁹ Coupling symptomatic investigation with a MVN-based diagnostic would allow medical staff to differentiate between pathogenic mycobacterial infections as most ManLAM-presenting pathogenic bacteria either cause cutaneous infection or pulmonary TB (lung infection) (Chan et al., 2015). Mycobacteria that cause cutaneous infections, such as M. leprae, induce illness that can be differentiated from M. tb empirically based on signature skin sores or lesions; therefore, they can be readily diagnosed without the need for LAM tests (Chan et al., 2015). Additionally, it is unnecessary to differentiate between pulmonary TB causing species, such as M. bovis, because they have the same or very similar treatment strategies to M. tb infection (CDC 2019). Hence, screening tests do not need to further differentiate between pathogenic mycobacteria infections to permit proper treatment.

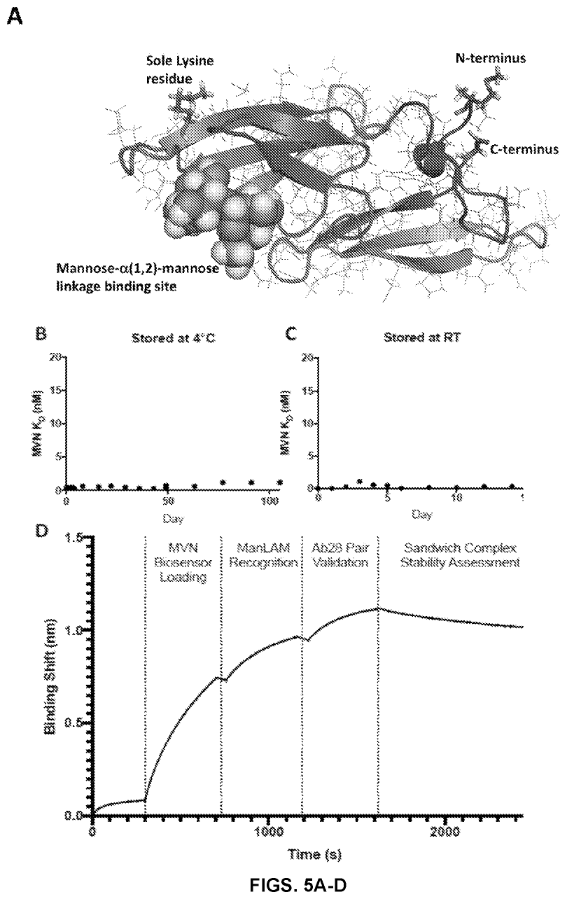

After confirming the high affinity and specificity of MVN to ManLAM, the greater utility of this capture reagent in a diagnostic assay was explored. MVN has several advantageous biophysical properties; it was expressed with a N-terminal hexa-histidine affinity tag and has a single intrinsic lysine residue which lends itself to facile bioconjugation techniques. Conjugating biotin to this residue enables the immobilization of MVN to any streptavidin coated substrate (Kehr et al., 2006; Koniev and Wagner, 2015). A PyMOL rendering of the MVN:Manα-(1,2)-Man crystal structure shows the location of the lysine residue and N-terminal hexa-histidine tag in proximity to the Manα-(1,2)-Man binding site (FIG. 5A) (Shahzad-ul-Hussan et al., 2017).

Stability is an important parameter to the utility of protein-based capture reagents, especially when employed in POC diagnostic tests. As a measure of stability, the binding affinity (K_{D}) of MVN conjugates, stored at room temp and at 4° C., was examined against H37Rv M. tb ManLAM over time. MVN remained stable at 4° C. for over 100 days (FIG. 5B), moreover MVN that was stored at room temperature (RT) for two weeks showed no evidence of degradation during the course of the experiment (FIG. 5C). Importantly, the thermal stability of MVN at room temperature will limit the need for cold-chain transportation of MVN-based POC tests to remote clinic sites in developing countries, further improving the accessibility of MVN-based POC diagnostic tests. Another practical advantage to using MVN is that, like in vitro mAb production, MVN can be produced via vector-based expression in E. coli, which can be readily scaled up in industrial bacterial bioreactors and is resistant to batch-to-batch variation. Furthermore, the stable expression of MVN in E. coli, a bacterium that multiplies every 20 minutes, and easily tolerates incubation and protein expression at lower temperatures, e.g., 18° C., means that MVN can be produced in large quantities at a significantly lower cost than recombinant proteins that have to be expressed in mammalian cells. In turn, the lower cost of production of MVN will translate to a lower cost per test, which is crucial to ensuring the successful rollout of POC diagnostic tests in developing countries.

In traditional “sandwich” style in vitro diagnostic tests, like ELISAs and LFAs, orthogonal binding pairs are employed to capture and detect biomarkers of interest. Pairing MVN with an orthogonal, high-affinity LAM capture element is therefore needed to detect ManLAM in these formats. Non-specific binding was observed between MVN and CS35, as well as MVN and CS40; additionally, supply constraints limited the use of Ab25 and Ab170. Therefore, Ab28 was chosen as the orthogonal LAM capture element for the binding pair and confirmed by bio-layer interferometry (FIG. 5D). MVN was not investigated as a self-binding pair (i.e., using MVN as both capture and detection elements) as MVN is known to bind to HIV-1 (Kehr et al., 2006). A MVN self-binding pair could display cross-reactivity with HIV-1 which would compromise the assay's specificity and sensitivity for M. tb ManLAM due to the high co-morbidity of HIV with TB Shahzad-ul-Hussan et al., 2017). Alternatively, when using MVN in a binding pair with an anti-LAM capture antibody, the antibody would provide specificity for LAM thereby eliminating cross-reactivity with HIV, while MVN would detect only ManLAM molecules among the captured LAM variants.

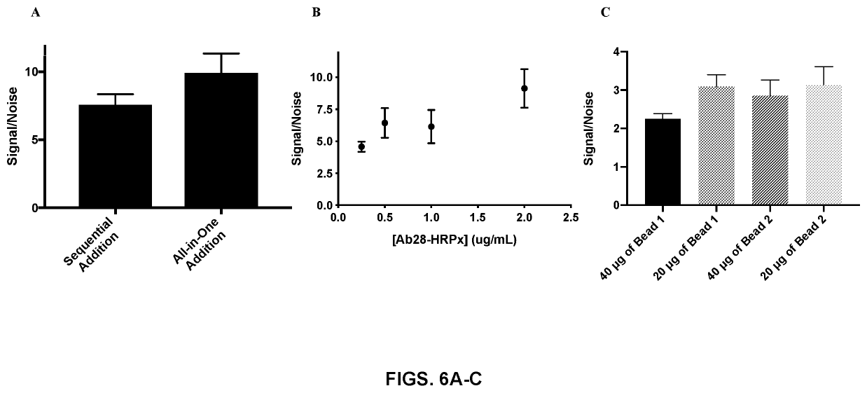

The model assay selected to showcase the high-affinity ManLAM-binding pair was an on-bead ELISA (OB-ELISA). The sub-picomolar binding affinity of MVN to ManLAM and opportunistic bioconjugation framework made it an ideal capture reagent, while the ubiquitous use of antibodies as detection conjugates in ELISAs prompted the selection of Ab28 as the detection conjugate (Damborsky et al., 2016; Astrom et al., 2017). The capture bead was generated by loading streptavidin magnetic Dynabeads with biotinylated MVN (Broger et al., 2019). An “all-in-one” OB-ELISA format was employed because it shortened overall assay time by 30+ minutes when compared to sequential addition without a significant impact on the signal-to-noise ratio (FIG. 6A) (Markwalter et al., 2016). The concentration of Ab28-HRP detection antibody was then optimized for the highest signal-to-noise ratio, where 2 μg/mL Ab28-HRP yielded choice results (FIG. 6B). In addition, number of beads used and MVN loading density were investigated. Bead 1 was functionalized with 60 μg MVN per 100 μL beads and Bead 2 was functionalized with 30 μg MVN per 100 μL bead. 40 μg of Bead 2 was selected as it produced a high signal-to-noise ratio and low variability (6.5%) over all concentrations tested (FIG. 6C). It is noteworthy that MVN has an approximately 10-fold lower molecular weight than IgG antibodies which could result in higher overall loading density of MVN. Further, for an equivalent bead surface loading density, the surface-bound MVN molecules would experience less steric hinderance and, hence, lower impairment on ManLAM-binding than the surface-bound anti-LAM IgG antibodies.

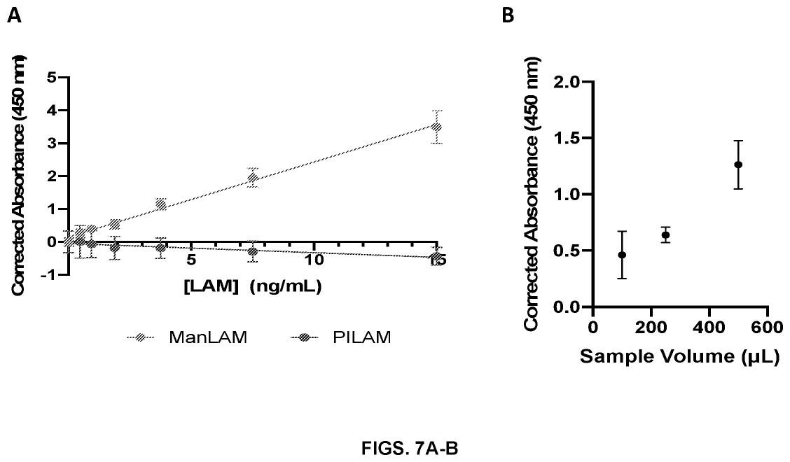

The OB-ELISA was then tested with mock patient samples consisting of ManLAM or PILAM spiked into pooled human urine. The urine was pooled from multiple normal doners and purchased from Innovative Research. A standard curve of ManLAM spiked into pooled human urine (n=7) was generated and the limit of detection was found to be 1.14 ng/mL with a variation of 6.18% (FIG. 7A). Importantly, no signal was observed when mock samples containing PILAM were tested, which provides strong evidence for the specificity of the inventors' MVN-based assay exclusively toward ManLAM. Lastly, larger urine sample volumes (250, 500 μL) were used in the OB-ELISA to show that the signal can be increased through volumetric enrichment by increasing the sample volume (FIG. 7B). A proportional increase in signal was observed with increasing sample volume; when a 250 μL sample was used, signal was enhanced by a factor of 1.4 compared to a theoretical enhancement of 2.7 and when a 500 μL sample was used, the resulting signal showed enhancement by a factor of 5.1 compared to the theoretical enhancement of 5. However, this finding is important as it shows that increasing sample volume leads to increased performance of the OB-ELISA. Large-volume urine samples are easily acquired—unlike other sample types (i.e., blood, sputum)—therefore, it would be feasible to use larger volumes of urine to improve the LOD of the OB-ELISA.

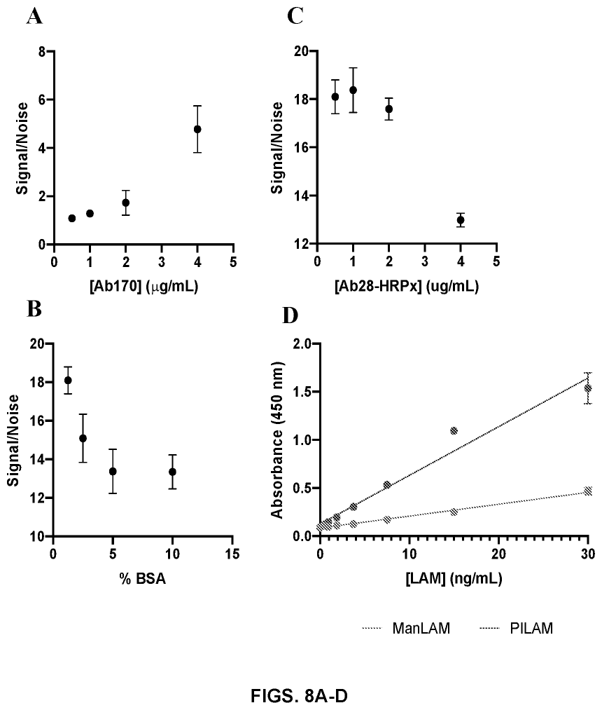

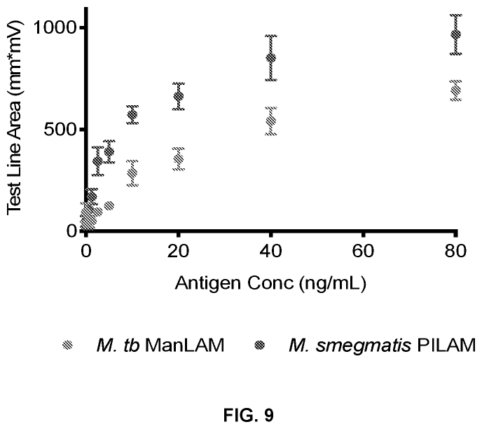

An antibody-based plate ELISA was also developed to compare the use of traditional molecular recognition elements (i.e., antibodies) to MVN (FIGS. 8A-D). In this assay, anti-LAM antibodies were used for both capture and detection. As expected, based on BLI results, this assay detects both ManLAM and PILAM spiked into pooled human urine with LODs of 729 pg/mL and 360 pg/mL, respectively. The inventors hypothesize that this assay is capable of detecting lower concentrations of PILAM than ManLAM due to the greater affinity of the anti-LAM antibodies for PILAM versus ManLAM. The antibody-based assay has a slightly lower LOD than the MVN-based OB-ELISA (1.14 ng/mL). They attribute this to the greater number of binding sites on the ManLAM arabinan branches for an anti-LAM antibody compared to the number of Manα-(1,2)-Man di-mannosyl cap binding sites for MVN. However, the MVN-based OB-ELISA has markedly improved specificity as no signal is observed when tested with PILAM.

Lastly, the sensitivity and specificity of the OB-ELISA were compared to the Alere LFA by testing the LFA with mock samples consisting of ManLAM or PILAM spiked into pooled human urine (FIG. S9). Both ManLAM and PILAM produce noticeable test line signal indicating that the Alere LFA is cross-reactive with PILAM, as expected based on the use of non-specific anti-LAM antibodies. Additionally, more intense test lines (greater test line area signal) were observed when LFAs were tested with PILAM which is in agreement with the results of the antibody-based ELISA. The visual LOD was determined to be 1.25 ng/mL ManLAM and 0.625 ng/mL PILAM which are similar to the reported values of 500-1000 pg/mL (MacLean and Pai, 2018; Garcia et al., 2019). The LOD of the OB-ELISA (1.14 ng/mL) falls in this range indicating that this assay is equally sensitive.

### Example 3—Discussion

POC diagnostic tests for the detection of the TB biomarker ManLAM in urine play an increasingly important role in HIV+TB coinfection. The inventors have employed the lectin MVN as the molecular recognition element to selectively detect ManLAM. Previous reports detail this lectin's high affinity and specificity for the Manα-(1,2)-Man di-mannosyl caps, but this is the first report that exploits this binding for the detection of ManLAM in TB diagnostic tests. BLI experiments showed that MVN has sub-picomolar binding affinity against ManLAM and stringent selectivity as it was shown that MVN does not bind PILAM, a variant of LAM found on non-pathogenic mycobacteria. In contrast, the employment of anti-LAM antibodies that bind to the arabinan branches of both ManLAM and PILAM leads to cross-reactivity and false positive results in diagnostic assays. Consequently, MVN is an ideal molecular recognition element for the development of POC TB diagnostic tests.

The inventors have developed an OB-ELISA that demonstrates the utility of MVN to selectively detect ManLAM in urine samples. As expected based on the BLI results, the assay exhibited a clear specificity for ManLAM versus PILAM, as well as a LOD of 1.14 ng/mL ManLAM, which falls between the LODs of an in-house antibody-based plate ELISA (729 pg/mL) and the Alere LFA (1.25 ng/mL). It is important to highlight that the plate ELISA and Alere LFA lack specificity for ManLAM and detect all variants of LAM. Further, the inventors have shown that the sample volume for the on-bead ELISA can be scaled up to increase the assay signal and, theoretically, decrease the LOD. Ultimately, the goal is to develop a test that is sensitive enough to detect low levels of ManLAM in HIV-negative, TB-positive patients. Future work includes optimizing the assay for use with large-volume urine samples and testing with clinical TB samples. The inventors have thus demonstrated the potential MVN has as a highly selective capture reagent for TB ManLAM in urine, paving the way for the development of next generation POC diagnostics that will serve as vital tools for future TB control, particularly in low-resource settings.

TABLE 1
Equilibrium binding affinity (as measured by dissociation constant)
of LAM recognition elements to different LAM variants.

| | Dissociation Constant (K_{D}) to Antigens | | |
|---|---|---|---|
| | H37Rv M. tb | M. leprae | M. smegmatis |
| | ManLAM | ManLAM | PILAM |
| MVN | <1 ± 9 pM | 74.4 ± 0.5 nM | N.B. |
| MVN-Biotin | 1850 ± 13 pM | 78.6 ± 0.5 nM | N.B. |
| FIND Ab25 | 1320 ± 16 pM | 967 ± 11 pM | 817 ± 18 pM |
| FIND Ab28 | 16.5 ± 0.4 nM | 10.7 ± 0.14 nM | 5.76 ± 0.11 nM |
| FIND Ab170 | 636 ± 11 pM | 118 ± 2 pM | <1 ± 2 pM |
| CS35 mAb | <1 ± 110 pM | <1.0 ± 60 pM | <1 ± 80 pM |
| CS40 mAb | 8740 ± 60 pM | Not tested | 113 ± 0.8 nM |
| Anti-LAM polyclonal | N.B. | N.B. | N.B. |
| (Rabbit) | | | |
| Anti-LAM polyclonal | N.B. | N.B. | N.B. |
| (Guinea pig) | | | |

N.B.—No binding observed

TABLE S1
Kinetic binding analysis of pooled fractions containing MVN-
His₆ to M. tb H37Rv ManLAM. The combined fraction Pools 2
and 3 exhibited higher binding affinity toward M. tb H37Rv
ManLAM and higher purity compared to Pools 1 and 4 (Figure
S3). Therefore, fraction Pools 2 and 3 were combined, and
used exclusively for further assay development.

| Fraction | Binding Affinity (K_{D}) to ManLAM | |
|---|---|---|
| 1 | Not tested | |
| 2 | 1.18 | nM |
| 3 | 61.3 | pM |
| 4 | 0.487 | nM |

### Example 4—Materials and Methods

Clinical subjects and samples. For this study, 58 urine samples were procured from the Foundation for Innovative New Diagnostics (FIND). These samples were previously collected from adults presenting at primary care sites in Cambodia, Peru, South Africa, and Vietnam with clinical symptoms of TB who had not yet been treated. Approval by local ethics committees and informed patient consent were obtained before enrolling the patients. No personally identifiable information was available to FIND or to the researchers. Urine and blood samples were collected at first contact with the patient and then centrifuged (at 200×g at 4° C. for 10 minutes), aliquoted, and frozen (at −80° C.) on the same day (typically within 4 hours). Due to reports of variation in the processing protocols for samples collected under different studies, the centrifugation step was repeated before analysis (Sigal et al., 2018). Sputum samples (typically two in the first 24 hours) were also collected from participants, decontaminated, and tested in up to six independent liquid cultures (MGIT; BD, Franklin Lakes, NJ, USA) and solid cultures (Lowenstein-Jensen medium). The presence of the M. tb complex in cultures was confirmed by Ziehl-Neelsen staining or auramine O fluorescence microscopy to identify acid-fast bacilli. HIV-status was determined by an HIV rapid test. The characteristics of the study population is summarized in Table 2.

TABLE 2
Sex, age, and country of origin information for the
study population stratified by TB- and HIV-status.

| | | TB-positive | | TB-negative | |
|---|---|---|---|---|---|
| | | HIV- | HIV- | HIV- | HIV- |
| Category | Number | positive | negative | positive | negative |
| All subjects | 58 | 16 | 18 | 12 | 12 |

Sex:

| | | | | | |
|---|---|---|---|---|---|
| Female | 25 | 7 | 2 | 9 | 7 |
| Male | 33 | 9 | 16 | 3 | 5 |

Age:

| | | | | | |
|---|---|---|---|---|---|
| 20-29 | 13 | 5 | 5 | 1 | 2 |
| 30-39 | 16 | 5 | 3 | 5 | 3 |
| 40-49 | 13 | 4 | 3 | 4 | 2 |
| 50-59 | 10 | 0 | 6 | 1 | 3 |
| 60+ | 6 | 2 | 1 | 1 | 2 |

Country:

| | | | | | |
|---|---|---|---|---|---|
| Cambodia | 2 | 0 | 2 | 0 | 0 |
| Peru | 14 | 2 | 4 | 1 | 7 |
| South Africa | 26 | 12 | 1 | 11 | 2 |
| Vietnam | 16 | 2 | 11 | 0 | 3 |

Urine sample preparation and testing. FIND samples were thawed and centrifuged for 10 minutes at 2000×g at 4° C. The liquid fraction was used moving forward. A positive (4 ng/mL) and negative ManLAM control were made from an aliquot of pooled human urine and centrifuged following the same procedure. All urine samples and controls were added to separate 3 kDa nominal molecular weight limit Amicon Ultra-4 Centrifugal Filter Units (Millipore Sigma: Cat. No. UFC800324). The centrifugal filter units were centrifuged with a swinging bucket rotor for 40 minutes at 4000×g and the concentrated sample was recovered with a pipette. The volume of the concentrated sample was then measured, and the sample was diluted with DI H₂O for a 14-fold concentration factor. Lastly, the diluted samples were heated at 85° C. for 10 minutes and the MVN-based OB-ELISA was performed as described in Chapter III, with FIND Ab194 used for detection due to supply constraints. A training video of this procedure was developed for use with collaborators. For each sample, the measured absorbance was normalized to the full process positive control run simultaneously to account for plate-to-plate variation.

Statistical analysis. All statistical analysis was performed using GraphPad Prism version 9.0.0 (86) for macOS, (GraphPad Software, San Diego, California USA, www.graphpad.com). Box-and-whisker plots were created to show the median and interquartile range for populations stratified by HIV-status, country of origin, and sex. One-tailed Mann Whitney tests were used to compare the mean absorbance values of TB-positive and TB-negative samples overall and in different sub-populations to determine if mannose caps are detectable on ManLAM in TB-positive samples with MVN. Spearman's correlation coefficient was calculated to study the correlation between absorbance and age for TB-positive and TB-negative samples. Approximate age was calculated from the YOB and day of enrollment, as age and day of birth information was not provided. Of note, this means all ages reported are ±1 year, but it is unlikely that it would change the general results presented.

Lastly, a receiver operating characteristic curve was produced based on the data to determine a cutoff for the OB-ELISA. Youden's J statistic and the Euclidian distance to the top-left corner were calculated for all points of the curve to determine the optimal cutoff value. The maximum value for Youden's J statistic was calculated to be 0.2353 for a cutoff value of 0.2611. This cutoff corresponded to a sensitivity of 24% and a specificity of 100%. The minimum distance was determined to be 0.6309 for a cutoff of 0.08516. This cutoff corresponded to a sensitivity of 68% and a specificity of 46%. The top-left cutoff was used moving forward as it balanced the trade-off between sensitivity and specificity. Using this cutoff value, sensitivity and specificity were calculated in each sub-population (HIV status, country of origin) with a 95% confidence interval calculated using a hybrid Wilson/Brown method. Positive and negative predictive values were not calculated due to the variation in TB prevalence by country (Dye et al., 1999). Positive and negative likelihood ratios, as well as the diagnostic odds ratio, were calculated for each sub-population.

### Example 5—Results

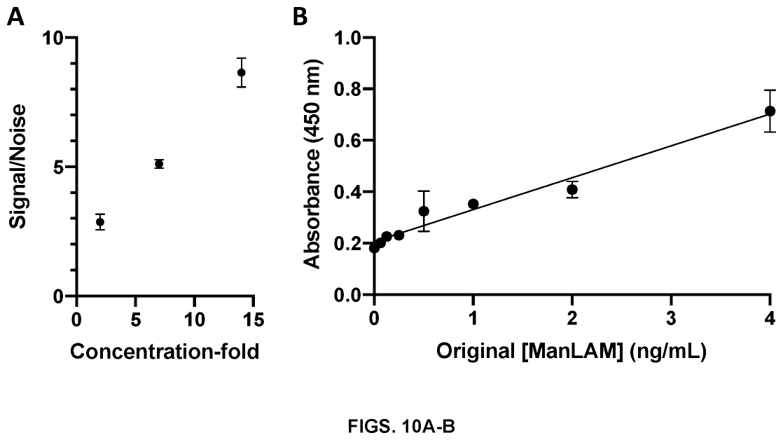

Clinical samples were tested with the MVN-based OB-ELISA. Due to the low concentrations of ManLAM in urine and the high background with the large-volume OB-ELISA, the samples were concentrated using Amicon Centrifugal filters. 2-fold, 7-fold, and 14-fold concentrated pooled human urine were compared (FIG. 10A). A 14-fold concentration factor was selected based on signal-to-noise. Then, a standard curve of 14-fold concentrated ManLAM spiked into pooled human urine (n=8) was used to determine the LOD, which was found to be 274 pg/mL (FIG. 10B).

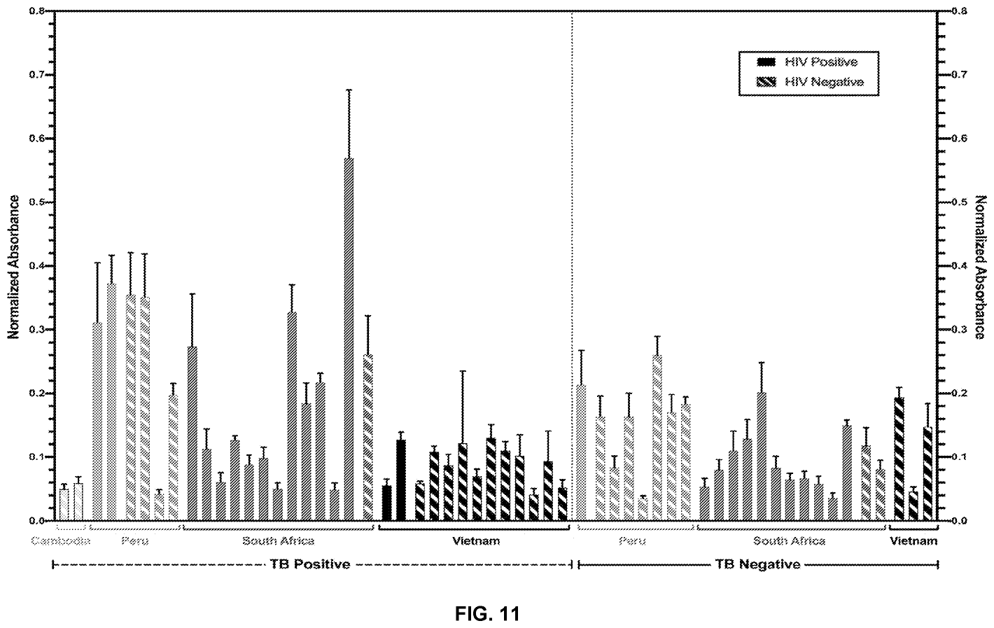

Next, clinical samples (n=58) were tested in 5 batches (Table 2). The samples originated from four countries: Cambodia (n=2), Peru (n=14), Vietnam (n=16), and Vietnam (n=16). Samples were defined as TB-positive if they had at least one positive culture. All of the TB-positive samples had positive microscopy results, as well. Additionally, samples were classified as HIV-positive (n=28) or HIV-negative (n=30) based on the result of an HIV rapid test. For each sample, the measured absorbance was normalized to the full process positive control run simultaneously to account for plate-to-plate variation (FIG. 11).

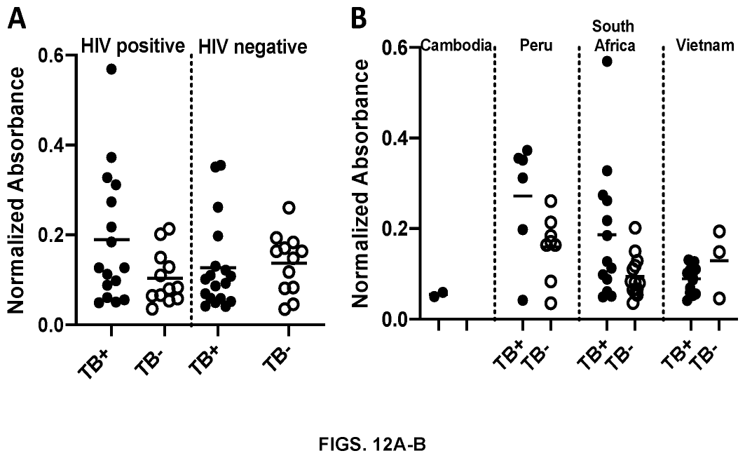

Mann Whitney tests were performed to determine the difference between TB-positive samples and TB-negative samples (Table 3). First, TB-positive and TB-negative samples were compared, and no significant difference was found in the means (p value=0.31). Then, the same analysis was performed on different sub-populations of the samples. There was also no significant difference between TB-positive and TB-negative samples for HIV-positive (p value=0.080) or HIV-negative (p value=0.22) samples. This was surprising as it is well-established that ManLAM concentrations are typically higher with HIV co-infection, resulting in increased detection ability in this sub-population (Paris et al., 2017). However, a significant difference in the means of TB-positive and TB-negative samples from Peru was observed (p value=0.030). No significant difference was observed in the means for samples from South Africa (p value=0.051) or Vietnam (p value=0.18). This analysis was not performed for samples from Cambodia as there were no TB-negative samples in that sub-population. Box-and-whisker plots were created to show the median and inter-quartile range for sub-populations by HIV-status and country of origin, as well (FIGS. 12A-B).

TABLE 3
Mann Whitney test p values for the difference between the mean
absorbance of TB-positive samples and TB-negative samples.

| Population | n, TB-positive | n, TB-negative | P value |
|---|---|---|---|
| All | 34 | 24 | 0.31 |
| HIV-positive | 16 | 12 | 0.080 |
| HIV-negative | 18 | 12 | 0.22 |
| Peru | 6 | 8 | 0.030 |
| South Africa | 13 | 13 | 0.051 |
| Vietnam | 13 | 3 | 0.18 |
| Female | 9 | 16 | 0.44 |
| Male | 25 | 8 | 0.35 |

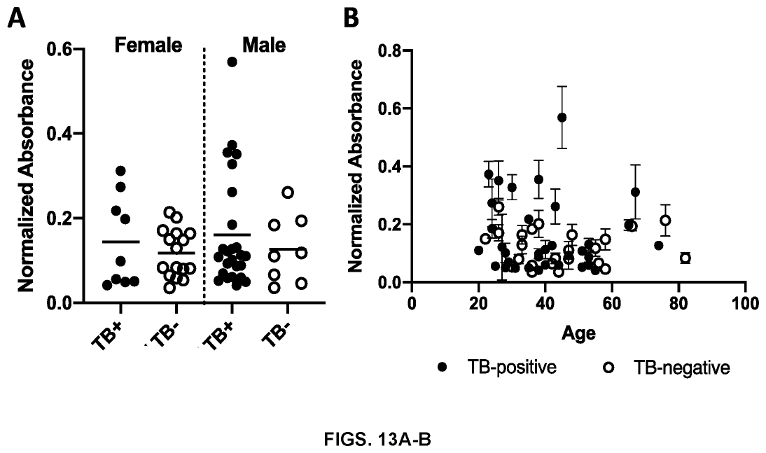

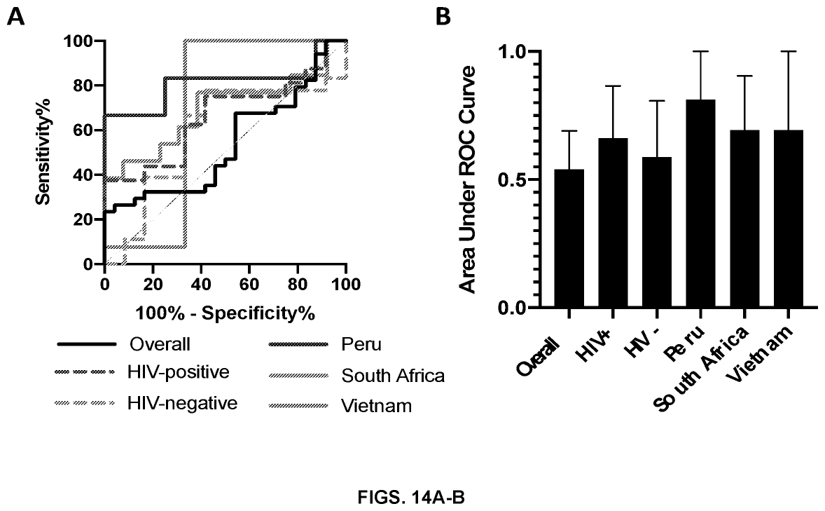

Association with biological sex and age was also tested. No significant difference was observed between TB-positive and TB-negative samples in female (p value=0.44) and male (p value=0.35) sub-populations by Mann-Whitney tests (FIG. 13A). Spearman's correlation coefficient was used to determine that there was no correlation between absorbance values and age for TB-positive (r=−0.10, p value=0.57) and TB-negative (r=−0.044, p value=0.84) samples (FIG. 13B).

Lastly, the MVN-based OB-ELISA was studied as a diagnostic test for comparison to other methods. A receiver operating characteristic curve was produced based on all the samples and for each sub-population stratified by HIV-status and country of origin (FIG. 14A). For each of these curves, the area under the curve was determined as a measure of diagnostic performance; interestingly, the area under the curve was larger in every sub-population (FIG. 14B).

The overall receiver operating characteristic curve was used to determine a cutoff for the OB-ELISA, and a cutoff value of 0.08516 was selected Using this cutoff value, the sensitivity was determined to be 68% and the specificity was found to be 46% (Table 4). In HIV-positive individuals, the sensitivity and specificity were slightly increased, while they were decreased in HIV-negative individuals. The sensitivity also varied widely by country, with Peru having the highest value and Vietnam having the lowest. This trend was reversed for specificity. Lastly, the diagnostics odd ratio was calculated for each of these sub-populations as a means of comparing sensitivity and specificity simultaneously (Fischer et al., 2003). The overall diagnostic odds ratio was calculated to be 1.8. However, this value was increased in samples from South Africa (5.3) and in HIV-positive individuals (4.2). There was no significant difference in samples from Peru (1.7) and the diagnostics odd ratio was reduced in samples from Vietnam (0.80) and in HIV-negative individuals (0.79).

TABLE 4
Sensitivity, specificity, positive and negative likelihood ratios
(LRs), and diagnostic odds ratio for all samples and sub-populations
stratified by HIV-status and country of origin.*

| | | | Positive | Negative | Diagnostic |
|---|---|---|---|---|---|
| Population | Sensitivity | Specificity | LR | LR | odds ratio |
| Overall | 68 | 46 | 1.2 | 0.71 | 1.8 |
| HIV+ | 75 | 58 | 1.8 | 0.43 | 4.2 |
| HIV− | 61 | 33 | 0.92 | 1.2 | 0.79 |
| Peru | 83 | 25 | 1.1 | 0.67 | 1.7 |
| South | 77 | 62 | 2.0 | 0.38 | 5.3 |
| Africa | | | | | |
| Vietnam | 62 | 33 | 0.92 | 1.2 | 0.80 |

*—The specificity indicated may be due to the same “cut offs” being used regardless of origin of samples. The AUC numbers in FIGS. 14A-B provide a better indication of diagnostic ability.

### Example 6—Discussion

Due to the low concentration of ManLAM in urine, an enrichment procedure was developed and optimized before analyzing clinical samples. The LOD deceased from 1.14 ng/mL to 274 pg/mL, a 4-fold improvement. However, this did not meet the expectation of a 14-fold improvement in the LOD in line with the concentration factor. It is hypothesized that this moderate increase is partially due to biomarker loss during the centrifugation step. Despite this, it was reasoned that ManLAM could be detected in over 50% of TB-positive samples, regardless of HIV-status, based on this LOD and prior studies.⁴¹ However, no significant difference was observed between mean absorbance values of TB-positive and TB-negative clinical samples.

There are many potential explanations for these results which will be explored from least to greatest impact. First, it is possible that MVN, like the mannose cap targeting antibody G3, is unable to detect in vivo ManLAM (Sigal et al., 2018). However, MVN is predicted to be more similar to the My2F12 antibody in terms on binding epitope (Choudhary et al., 2018; Shahzad-ul-Hussan et al., 2011). As previously mentioned, both G3 and My2F12 were found to bind strongly to di-mannose capping motifs, but G3 exhibited a weak affinity for mono-mannose caps and My2F12 showed a weak affinity for tri-mannose caps (Choudhary et al., 2018). MVN was found to bind to both di-mannose and tri-mannose structures, with a higher affinity for larger structures like the HIV glycan Man₉ (Shahzad-ul-Hussan et al., 2011). However, MVN has not been tested in a glycan array with LAM-specific structures, limiting further comparisons.

A second hypothesis is that the difference in binding results between the three molecular recognition elements is due to the clinical samples being tested. This study and the G3 antibody study both utilized urine samples from the Foundation for Innovative New Diagnostics. (FIND) (Sigal et al., 2018). Further, the G3 study mainly tested samples from Peru, South Africa, and Vietnam, with a minor contribution of samples from Bangladesh (Sigal et al., 2018). Thus, there is likely considerable overlap in confounding factors between the two studies. In contrast, the My2F12 study utilized samples from Georgia (Chan et al., 2015). Specifically, the sample processing methods and storage conditions for these samples differ. The standard protocol for FIND is to centrifuge urine samples at 200×g at 4° C. for 10 minutes. In contrast, the samples used in the My2F12 study were heated at 95° C. for 30 minutes in a drying oven (Chan et al., 2015). However, in this study centrifugation was necessary to concentrate the samples to allow for ManLAM detection. Further, the arabinose backbone and MTX modifications have been detected after centrifugation weakening this hypothesis (Sigal et al., 2018). Another difference between the samples is storage temperature; FIND samples are frozen at −80° C., while the My2F12 samples were frozen at −20° C. (Chan et al., 2015). One study reported a roughly 50% loss of ManLAM detection by the Alere LFA after freezing for one day at −70° C. (Connelly et al., 2019). A study with the Fuji-LAM test disagreed with this result, showing no loss after one month at −20° C. (Broger et al., 2020). However, it is possible that lower temperatures have a negative impact on ManLAM detection ability.

Additionally, the samples tested in this study, the G3 study, and the My2F2 study differed in geographic origin which could contribute to concentration differences in the ManLAM antigen. This hypothesis is supported by the observed variable results by country, with a significant difference in mean absorbance values only observed in Peruvian samples. Notably, this cannot be attributed to HIV as only 21% of all samples from Peru and 33% of TB-positive samples from Peru were HIV-positive. Previously reported differences in ManLAM concentration by geographic location, that cannot be explained by HIV status, were mainly attributed to the quality of the urine sample and glycosuria, which can be caused by diabetes (Magni et al., 2020). However, information on diabetes status was not collected for these samples. Additionally, other risk factors—such as nutritional status, alcohol use, and tobacco use—have been shown to contribute to TB progression, and thus ManLAM detectability (Narasihman et al., 2013; Martins-Melo et al., 2020). Without more information, it is impossible to say whether these risk factors are causing the observed variation in ManLAM concentration.

Another explanation is that in vivo ManLAM could be endogenously modified in some populations contributing to geographic variation. De et al. has hypothesized that “host enzymes either partially degrade the complex glycoform of ManLAM or modulate the structure of the sugars present at the caps that are characteristic of pathogenic strains of mycobacteria.” (De et al., 2020). Human mannosidase enzymes in the endoplasmic reticulum and Golgi complex are capable of degrading the α-(1,2) mannose linkages in the HIV glycan Man₉ (al Daher et al., 1991; Lal et al., 1998; Tremblay et al., 2000; Zhou et al., 2015; Crispin et al., 2007; Jan et al., 2019; Sobala et al., 2020). Notably, the α-(1,2)-mannosidase-IA has been shown to preferentially degrade the Man₉ D1 mannose residue first, which corresponds to the MVN binding site on Man₉ (Lal et al., 1998; Jan et al., 2019). Further, these enzymes have been shown to be up- or down-regulated in response to disease state, including multiple sclerosis and various cancers (Mkhikian et al., 2011; Tu et al., 2017; Legler et al., 2018). However, no information was found regarding tuberculosis representing an area for further study. Interestingly, increased lysosomal α-mannosidase activity has been reported in people with diabetes, a common co-morbidity with TB (Goi et al., 1987; Waters et al., 1992; Harries et al., 2013). Of the three countries of origin analyzed in this study, Vietnam has the highest prevalence of diabetes, which supports this reasoning (Lin et al., 2020).

Lastly, epitope differences in ManLAM, particularly in the mannose caps or MTX residues, caused by pathogenic characteristics could explain the variable results by geography. Past results have shown that ManLAM epitopes vary in their discrimination performance by country (Magni et al., 2020). One potential cause of ManLAM epitope differences is TB strain, which varies geographically (Broger et al., 2020; Weins et al., 2018). Globally, the Euro-American lineage 4 is the most common, representing the dominant lineage in Georgia (61.3%), Peru (79.6%), and South Africa (72.2%) (Weins et al., 2018). However, lineage 4 only represents 7.6% of the M. tb burden in Vietnam with the East Asian lineage 2 (37.4%) and Indo-Oceanic lineage 1 (24.9%) constituting the majority Weins et al., 2018). Reports from other have indicated significant variation in low molecular weight lipids between in vitro M. tb strains from lineages 2, 3, and 4 (J. Blackburn, personal communication). However, this lipidomic study did not attempt to detect ManLAM due the complexity of the molecule. In contrast, a separate mass spectrometry study of ManLAM was able to detect mannose caps and MTX modifications in a clinical sample from Peru and an in vitro lineage 2 strain; however, the relative abundance of various mannose cap structures varied between the two sources (De et al., 2020). In my professional opinion, this hypothesis that the capping motifs or their relative abundance could vary by lineage is the most likely explanation of the observed geographic variation as ManLAM is known to be an extremely heterogenous molecule (Broger et al., 2020). Further research into the exact binding site of MVN and the performance of the OB-ELISA by M. tb lineage is needed as this hypothesis could have important ramifications on the ability to diagnose TB with universal LAM detection tests.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
Abbondanzo et al., Am. J. Pediatr. Hematol. Oncol. 12(4): 480-489, 1990.
Allred et al., Arch. Surg. 125(1), 107-113: 1990.
Atherton et al., Biol. of Reproduction 32: 155-171, 1985.
Barzon et al., Euro Surveill. 11: 21(32), 2016.
Beltramello et al., Cell Host Microbe 8: 271-283, 2010.
Brown et al., J. Immunol. Meth. 130(1): 111-121, 1990.
Capaldi et al., Biochem. Biophys. Res. Comm. 74(2):4 25-433, 1977.
De Jager et al., Semin. Nucl. Med. 23(2): 165-179, 1993.
Dholakia et al., J. Biol. Chem. 264: 20638-20642, 1989.
Diamond et al., J. Virol. 77: 2578-2586, 2003.
Doolittle and Ben-Zeev, Methods Mol. Biol. 109: 215-237, 1999.
Duffy et al., N. Engl. J. Med. 360: 2536-2543, 2009.
Gefter et al., Somatic Cell Genet. 3: 231-236, 1977.
Gornet et al., Semin Reprod Med. 34(5): 285-292, 2016.
Gulbis and Galand, Hum. Pathol. 24(12): 1271-1285, 1993.
Halfon et al., PLoS ONE 5(5): e10569, 2010
Hessell et al., Nature 449: 101-104, 2007.
Khatoon et al., Ann. of Neurology 26: 210-219, 1989.
King et al., J. Biol. Chem. 269: 10210-10218, 1989.
Kyte and Doolittle, J. Mol. Biol. 157(1): 105-132, 1982.
Mansuy et al., Lancet Infect Dis. 16(10): 1106-7, 2016.
Nakamura et al., In: Enzyme Immunoassays: Heterogeneous and Homogeneous Systems, Chapter 27, 1987.
O'Shannessy et al., J. Immun. Meth. 99: 153-161, 1987.
Persic et al., Gene 187: 1, 1997
Potter and Haley, Meth. Enzymol. 91: 613-633, 1983.
Purpura et al., Lancet Infect Dis. 16(10): 1107-8, 2016.
Remington's Pharmaceutical Sciences, 15th Ed., 3: 624-652, 1990.
Tang et al., J. Biol. Chem., 271: 28324-28330, 1996.
Wawrzynczak & Thorpe, In: Immunoconjugates, Antibody Conjugates In Radioimaging And Therapy Of Cancer, Vogel (Ed.), NY, Oxford University Press, 28, 1987.
Yu et al., J Immunol Methods 336: 142-151, 2008.
World Health Organization, Global Tuberculosis Report 2018, World Health Organization, S.l., 2018.
M. Pai, M. P. Nicol and C. C. Boehme, Microbiol. Spectr., DOI:10.1128/microbiolspec.TBTB2-0019-2016.
E. MacLean, K. Saravu and M. Pai, Curr. Opin. HIV AIDS, DOI:10.1097/COH.0000000000000512, 2019.
C. K. Kwan and J. D. Ernst, Clin. Microbiol. Rev., 2011, 24, 351-376.
A. D. Harries and A. M. V. Kumar, Diagn. Basel Switz., DOI:10.3390/diagnostics8040078, 2018.
R. Wood, K. Racow, L. G. Bekker, K. Middelkoop, M. Vogt, B. N. Kreiswirth and S. D. Lawn, BMC Infect. Dis., 2012, 12, 47.
S. D. Lawn, BMC Infect. Dis., 2012, 12, 103.
K. Dheda, V. Davids, L. Lenders, T. Roberts, R. Meldau, D. Ling, L. Brunet, R. van Zyl Smit, J. Peter, C. Green, M. Badri, L. Sechi, S. Sharma, M. Hoelscher, R. Dawson, A. Whitelaw, J. Blackburn, M. Pai and A. Zumla, PloS One, 2010, 5, e9848.
S. Sarkar, X. L. Tang, D. Das, J. S. Spencer, T. L. Lowary and M. R. Suresh, PloS One, 2012, 7, e32340.
J. Minion, E. Leung, E. Talbot, K. Dheda, M. Pai and D. Menzies, Eur. Respir. J., 2011, 38, 1398-1405.
P. Sarkar, D. Biswas, G. Sindhwani, J. Rawat, A. Kotwal and B. Kakati, Postgrad. Med. J., 2014, 90, 155-163.
C. Boehme, E. Molokova, F. Minja, S. Geis, T. Loscher, L. Maboko, V. Koulchin and M. Hoelscher, Trans. R. Soc. Trop. Med. Hyg., 2005, 99, 893-900.
A. K. Mishra, N. N. Driessen, B. J. Appelmelk and G. S. Besra, FEMS Microbiol. Rev., 2011, 35, 1126-1157.
J. Nigou, M. Gilleron and G. Puzo, Biochimie, 2003, 85, 153-166.
J. Nigou, A. Vercellone and G. Puzo, J. Mol. Biol., 2000, 299, 1353-1362.
J. B. Torrelles, K.-H. Khoo, P. A. Sieling, R. L. Modlin, N. Zhang, A. M. Marques, A. Treumann, C. D. Rithner, P. J. Brennan and D. Chatterjee, J. Biol. Chem., 2004, 279, 41227-41239.
C. E. Chan, S. Götze, G. T. Seah, P. H. Seeberger, N. Tukvadze, M. R. Wenk, B. J. Hanson and P. A. MacAry, Sci. Rep., 2015, 5, 10281.
D. Kaur, T. L. Lowary, V. D. Vissa, D. C. Crick and P. J. Brennan, Microbiol. Read. Engl., 2002, 148, 3049-3057.
T. Broger, B. Sossen, E. du Toit, A. D. Kerkhoff, C. Schutz, E. Ivanova Reipold, A. Ward, D. A. Barr, A. Macé, A. Trollip, R. Burton, S. Ongarello, A. Pinter, T. L. Lowary, C. Boehme, M. P. Nicol, G. Meintjes and C. M. Denkinger, Lancet Infect. Dis., DOI:10.1016/51473-3099(19)30001-5, 2019.
M. Kawasaki, C. Echiverri, L. Raymond, E. Cadena, E. Reside, M. T. Gler, T. Oda, R. Ito, R. Higashiyama, K. Katsuragi and Y. Liu, PLOS Med., 2019, 16, e1002780.
J.-C. Kehr, Y. Zilliges, A. Springer, M. D. Disney, D. D. Ratner, C. Bouchier, P. H. Seeberger, N. T. de Marsac and E. Dittmann, Mol. Microbiol., 2006, 59, 893-906.
D. Huskens, G. Ferir, K. Vermeire, J.-C. Kehr, J. Balzarini, E. Dittmann and D. Schols, J. Biol. Chem., 2010, 285, 24845-24854.
S. Shahzad-ul-Hussan, E. Gustchina, R. Ghirlando, G. M. Clore and C. A. Bewley, J. Biol. Chem., 2011, 286, 20788-20796.
D. Goletti, E. Petruccioli, S. A. Joosten and T. H. M. Ottenhoff, Infect. Dis. Rep., DOI:10.4081/idr.2016.6568.
N. S. Lipman, L. R. Jackson, L. J. Trudel and F. Weis-Garcia, ILAR J., 2005, 46, 258-268.
A. G. Amin, P. De, J. S. Spencer, P. J. Brennan, J. Daum, B. G. Andre, M. Joe, Y. Bai, L. Laurentius, M. D. Porter, W. J. Honnen, A. Choudhary, T. L. Lowary, A. Pinter and D. Chatterjee, Tuberculosis, 2018, 111, 178-187.
A. Treumann, F. Xidong, L. McDonnell, P. J. Derrick, A. E. Ashcroft, D. Chatterjee and S. W. Homans, J. Mol. Biol., 2002, 316, 89-100.
S. Shahzad-Ul-Hussan, M. Sastry, T. Lemmin, C. Soto, S. Loesgen, D. A. Scott, J. R. Davison, K. Lohith, R. O'Connor, P. D. Kwong and C. A. Bewley, Chembiochem Eur. J. Chem. Biol., 2017, 18, 764-771.
Y. Q. Min, X. C. Duan, Y. D. Zhou, A. Kulinich, W. Meng, Z. P. Cai, H. Y. Ma, L. Liu, X. L. Zhang and J. Voglmeir, Biosci. Rep., DOI:10.1042/BSR20170015.
Fact Sheets|General|Mycobacterium bovis (Bovine Tuberculosis) in Humans|TB|CDC, world-wide-web at cdc.gov/tb/publications/factsheets/general/mbovis.htm, (accessed Jul. 15, 2019).
O. Koniev and A. Wagner, Chem. Soc. Rev., 2015, 44, 5495-5551.
P. Damborský, K. M. Koczula, A. Gallotta and J. Katrlík, The Analyst, 2016, 141, 6444-6448.
E. Åström, P. Stål, R. Zenlander, P. Edenvik, C. Alexandersson, M. Haglund, I. Rydén and P. Påhlsson, PLOS ONE, 2017, 12, e0173897.
C. F. Markwalter, K. M. Ricks, A. L. Bitting, L. Mudenda and D. W. Wright, Talanta, 2016, 161, 443-449.
E. MacLean and M. Pai, Clin. Chem., 2018, 64, 1133-1135.
J. I. García, H. V. Kelley, J. Meléndez, R. A. A. de León, A. Castillo, S. Sidiki, K. A. Yusoof, E. Nunes, C. L. Téllez, C. R. Mejía-Villatoro, J. Ikeda, A. L. García-Basteiro, S.-H. Wang and J. B. Torrelles, Sci. Rep., 2019, 9, 1-6.
Dye, C.; Scheele, S.; >Dolin, P.; Pathania, V.; Raviglione, M. C.; for the WHO Global Surveillance and Monitoring Project. Global Burden of Tuberculosis: Estimated Incidence, Prevalence, and Mortality by Country. JAMA 1999, 282 (7), 677-686.
Fischer, J. E.; Bachmann, L. M.; Jaeschke, R. A Readers' Guide to the Interpretation of Diagnostic Test Properties: Clinical Example of Sepsis. Intensive Care Med. 2003, 29 (7), 1043-1051.
Connelly, J. T.; Grant, B.; Munsamy, V.; Pym, A.; Somoskovi, A. Lipoarabinomannan Point-of-Care Tests: Evaluation with Fresh Samples Needed. Lancet Infect. Dis. 2019, 19 (10), 1053.
Broger, T.; Muyoyeta, M.; Kerkhoff, A. D.; Denkinger, C. M.; Moreau, E. Tuberculosis Test Results Using Fresh versus Biobanked Urine Samples with FujiLAM. Lancet Infect. Dis. 2020, 20 (1), 22-23.
Magni, R.; Rruga, F.; Alsaab, F.; Sharif, S.; Howard, M.; Espina, V.; Kim, B.; Lepene, B.; Lee, G.; Alayouni, M. A.; Steinberg, H.; Araujo, R.; Kashanchi, F.; Riccardi, F.; Morreira, S.; Araujo, A.; Poli, F.; Jaganath, D.; Semitala, F. C.; Worodria, W.; Andama, A.; Choudhary, A.; Honnen, W. J.; Petricoin, E. F.; Cattamanchi, A.; Colombatti, R.; de Waard, J. H.; Oberhelman, R.; Pinter, A.; Gilman, R. H.; Liotta, L. A.; Luchini, A. Lipoarabinomannan Antigenic Epitope Differences in Tuberculosis Disease Subtypes. Sci. Rep. 2020, 10 (1), 13944.
Narasimhan, P.; Wood, J.; MacIntyre, C. R.; Mathai, D. Risk Factors for Tuberculosis. Pulm. Med. 2013, 2013.
Martins-Melo, F. R.; Bezerra, J. M. T.; Barbosa, D. S.; Carneiro, M.; Andrade, K. B.; Ribeiro, A. L. P.; Naghavi, M.; Werneck, G. L. The Burden of Tuberculosis and Attributable Risk Factors in Brazil, 1990-2017: Results from the Global Burden of Disease Study 2017. Popul. Health Metr. 2020, 18 (1), 10.
al Daher, S.; de Gasperi, R.; Daniel, P.; Hall, N.; Warren, C. D.; Winchester, B. The Substrate-Specificity of Human Lysosomal Alpha-D-Mannosidase in Relation to Genetic Alpha-Mannosidosis. Biochem. J. 1991, 277 (Pt 3), 743-751.
Lal, A.; Pang, P.; Kalelkar, S.; Romero, P. A.; Herscovics, A.; Moremen, K. W. Substrate Specificities of Recombinant Murine Golgi Alpha1, 2-Mannosidases IA and IB and Comparison with Endoplasmic Reticulum and Golgi Processing Alpha1,2-Mannosidases. Glycobiology 1998, 8 (10), 981-995.
Tremblay, L. O.; Herscovics, A. Characterization of a CDNA Encoding a Novel Human Golgi A1,2-Mannosidase (IC) Involved in N-Glycan Biosynthesis. J. Biol. Chem. 2000, 275 (41), 31655-31660.
Zhou, T.; Frabutt, D. A.; Moremen, K. W.; Zheng, Y.-H. ERManI (Endoplasmic Reticulum Class I α-Mannosidase) Is Required for HIV-1 Envelope Glycoprotein Degradation via Endoplasmic Reticulum-Associated Protein Degradation Pathway. J. Biol. Chem. 2015, 290 (36), 22184-22192.
Crispin, M.; Aricescu, A. R.; Chang, V. T.; Jones, E. Y.; Stuart, D. I.; Dwek, R. A.; Davis, S. J.; Harvey, D. J. Disruption of α-Mannosidase Processing Induces Non-Canonical Hybrid-Type Glycosylation. FEBS Letters 2007, 581 (10), 1963-1968.
Jan, M.; Upadhyay, C.; Hioe, C. E. HIV-1 Envelope Glycan Composition as a Key Determinant of Efficient Virus Transmission via DC-SIGN and Resistance to Inhibitory Lectins. iScience 2019, 21, 413-427.
Sobala, Ł. F.; Fernandes, P. Z.; Hakki, Z.; Thompson, A. J.; Howe, J. D.; Hill, M.; Zitzmann, N.; Davies, S.; Stamataki, Z.; Butters, T. D.; Alonzi, D. S.; Williams, S. J.; Davies, G. J. Structure of Human Endo-α-1,2-Mannosidase (MANEA), an Antiviral Host-Glycosylation Target. Proc. Natl. Acad. Sci. 2020, 117 (47), 29595-29601.
Mkhikian, H.; Grigorian, A.; Li, C. F.; Chen, H. L.; Newton, B.; Zhou, R. W.; Beeton, C.; Torossian, S.; Tatarian, G. G.; Lee, S. U.; Lau, K.; Walker, E.; Siminovitch, K. A.; Chandy, K. G.; Yu, Z.; Dennis, J. W.; Demetriou, M. Genetics and the Environment Converge to Dysregulate N-Glycosylation in Multiple Sclerosis. Nat. Commun. 2011, 2, 334.
Tu, H.; Hsiao, Y. C.; Yang, W. Y.; Tsai, S. L.; Lin, H. K.; Liao, C. Y.; Lu, J. W.; Chou, Y. T.; Wang, H. D.; Yuh, C. H. Up-Regulation of Golgi α-Mannosidase IA and down-Regulation of Golgi α-Mannosidase IC Activates Unfolded Protein Response during Hepatocarcinogenesis. Hepatol. Commun. 2017, 1 (3), 230-247.
Legler, K.; Rosprim, R.; Karius, T.; Eylmann, K.; Rossberg, M.; Wirtz, R. M.; Müller, V.; Witzel, I.; Schmalfeldt, B.; Milde-Langosch, K.; Oliveira-Ferrer, L. Reduced Mannosidase MAN1A1 Expression Leads to Aberrant N-Glycosylation and Impaired Survival in Breast Cancer. Br. J. Cancer 2018, 118 (6), 847-856.
Goi, G.; Lombardo, A.; Fabi, A.; Burlina, A. B.; Segalini, G.; Guagnellini, E.; Tettamanti, G. Serum Enzymes of Lysosomal Origin as Indicators of the Metabolic Control in Non-Insulin-Dependent Diabetics. Acta Diabetol. Lat/1987, 24 (4), 331-340.
Waters, P. J.; Flynn, M. D.; Corrall, R. J.; Pennock, C. A. Increases in Plasma Lysosomal Enzymes in Type 1 (Insulin-Dependent) Diabetes Mellitus: Relationship to Diabetic Complications and Glycaemic Control. Diabetologia 1992, 35 (10), 991-995.
Harries, A. D.; Satyanarayana, S.; Kumar, A. M. V.; Nagaraja, S. B.; Isaakidis, P.; Malhotra, S.; Achanta, S.; Naik, B.; Wilson, N.; Zachariah, R.; Lönnroth, K.; Kapur, A. Epidemiology and Interaction of Diabetes Mellitus and Tuberculosis and Challenges for Care: A Review. Public Health Action 2013, 3 (1), 3-9.
Lin, X.; Xu, Y.; Pan, X.; Xu, J.; Ding, Y.; Sun, X.; Song, X.; Ren, Y.; Shan, P.-F. Global, Regional, and National Burden and Trend of Diabetes in 195 Countries and Territories: An Analysis from 1990 to 2025. Sci. Rep. 2020, 10 (1), 14790.
Wiens, K. E.; Woyczynski, L. P.; Ledesma, J. R.; Ross, J. M.; Zenteno-Cuevas, R.; Goodridge, A.; Ullah, I.; Mathema, B.; Djoba Siawaya, J. F.; Biehl, M. H.; Ray, S. E.; Bhattacharjee, N. V.; Henry, N. J.; Reiner, R. C.; Kyu, H. H.; Murray, C. J. L.; Hay, S. I. Global Variation in Bacterial Strains That Cause Tuberculosis Disease: A Systematic Review and Meta-Analysis. BMC Med. 2018, 16 (1), 196.
Choudhary, A.; Patel, D.; Honnen, W.; Lai, Z.; Prattipati, R. S.; Zheng, R. B.; Hsueh, Y.-C.; Gennaro, M. L.; Lardizabal, A.; Restrepo, B. I.; Garcia-Viveros, M.; Joe, M.; Bai, Y.; Shen, K.; Sahloul, K.; Spencer, J. S.; Chatterjee, D.; Broger, T.; Lowary, T. L.; Pinter, A. Characterization of the Antigenic Heterogeneity of Lipoarabinomannan, the Major Surface Glycolipid of Mycobacterium tuberculosis, and Complexity of Antibody Specificities toward This Antigen. J. Immunol. 2018, 200 (9), 3053-3066.
Sigal, G. B.; Pinter, A.; Lowary, T. L.; Kawasaki, M.; Li, A.; Mathew, A.; Tsionsky, M.; Zheng, R. B.; Plisova, T.; Shen, K.; Katsuragi, K.; Choudhary, A.; Honnen, W. J.; Nahid, P.; Denkinger, C. M.; Broger, T. A Novel Sensitive Immunoassay Targeting the 5-Methylthio-d-Xylofuranose-Lipoarabinomannan Epitope Meets the WHO's Performance Target for Tuberculosis Diagnosis. J. Clin. Microbiol. 2018, 56 (12).
Shahzad-ul-Hussan, S.; Gustchina, E.; Ghirlando, R.; Clore, G. M.; Bewley, C. A. Solution Structure of the Monovalent Lectin Microvirin in Complex with Manα(1-2)Man Provides a Basis for Anti-HIV Activity with Low Toxicity. J. Biol. Chem. 2011, 286 (23), 20788-96.
Chan, C. E.; Götze, S.; Seah, G. T.; Seeberger, P. H.; Tukvadze, N.; Wenk, M. R.; Hanson, B. J.; MacAry, P. A. The Diagnostic Targeting of a Carbohydrate Virulence Factor from M. tuberculosis. Sci. Rep. 2015, 5, 10281.
De, P.; Shi, L.; Boot, C.; Ordway, D.; McNeil, M.; Chatterjee, D. Comparative Structural Study of Terminal Ends of Lipoarabinomannan from Mice Infected Lung Tissues and Urine of a Tuberculosis Positive Patient. ACS Infect. Dis. 2020, 6 (2), 291-301.
Paris, L.; Magni, R.; Zaidi, F.; Araujo, R.; Saini, N.; Harpole, M.; Coronel, J.; Kirwan, D. E.; Steinberg, H.; Gilman, R. H.; Petricoin, E. F.; Nisini, R.; Luchini, A.; Liotta, L. Urine Lipoarabinomannan Glycan in HIV-Negative Patients with Pulmonary Tuberculosis Correlates with Disease Severity. Sci. Transl. Med. 2017, 9 (420).

What is claimed is:

1. A method of diagnosing a Mycobacterium tuberculosis infection in a subject comprising:
(a) providing a biological sample from a subject;
(b) contacting said biological sample with microvirin-N (MVN); and
(c) detecting binding of microvirin-N to mannose-capped lipoarabinomannan (ManLAM) in said biological sample,
wherein binding of MVN to mannose-capped lipoarabinomannan (ManLAM) in said biological sample indicates that said subject is infected with Mycobacterium tuberculosis.

2. The method of claim 1, wherein said biological sample is a fluid sample, such as blood, sputum, or urine.

3. The method of claim 1, wherein MVN is bound to a support.

4. (canceled)

5. The method of claim 3, wherein ManLAM is detected when bound to said support to MVN with a lipoarabinomannan (LAM)-binding agent.

6. The method of claim 5, wherein said LAM binding agent is a monoclonal antibody (mAb), such as a mAb binding to a D-mannan core structure or a D-arabinan branch structure, such as a tetra-arabinoside motif or hexa-arabinan motif.

7. The method of claim 5, wherein said LAM-binding agent comprises a detectable label.

8. The method of claim 1, wherein said subject is suspected as having a Mycobacterium tuberculosis infection or has been exposed to a patient with a Mycobacterium tuberculosis infection.

9. The method of claim 1, wherein said subject is at increased risk of contracting a Mycobacterium tuberculosis infection as compared to populational average.

10. The method of claim 1, further comprising performing a positive control reaction for binding of MVN to ManLAM and/or further comprising performing a negative control reaction for binding of MVN to ManLAM.

11. The method of claim 1, further comprising performing steps (a)-(c) a second time on said sample or on a different sample from said subject.

12. (canceled)

13. The method of claim 1, wherein the limit of detection of ManLAM is about 500-1500 pg/ml or about 6251250 pg/ml.

14 (canceled)

15. The method of claim 1, wherein the limit of detection of ManLAM is about 1400 pg/ml.

16. An isolated complex comprising microvirin-N bound to (a) a support and (b) mannose-capped lipoarabinomannan (ManLAM).

17. The isolated complex of claim 16, further bound to an antibody that is selective for binding to lipoarabinomannan (LAM), such as a labeled anti-LAM antibody.

18. The isolated complex of claim 17, wherein the anti-LAM antibody is a mAb binding to a D-mannan core structure or a D-arabinan branch structure, such as wherein the D-mannan core structure or D-arabinan branch structure is a tetra-arabinoside motif or hex-arabinan motif.

19 (canceled)

20. The isolated complex of claim 16, wherein the support is a bead, such as a magnetic bead, a microtiter well, a dipstick, a filter, or a membrane.

21. A kit comprising microvirin-N bound to a support.

22. The kit of claim 21, further comprising an antibody that is selective for binding to lipoarabinomannan (LAM), such as a labeled anti-LAM antibody.

23. The kit of claim 22, wherein the anti-LAM antibody is a mAb binding to a D-mannan core structure or a D-arabinan branch structure, such as a tetra-arabinoside motif or hexa-arabinan motif.

24. (canceled)

25. The kit of claim 21, further comprising one or more of (i) instructions for performing an assay for detecting mannose-capped lipoarabinomannan in a sample, (ii) mannose-capped lipoarabinomannan, (iii) a reagent comprising a D-mannan core structure or a D-arabinan branch structure, such as a tetra-arabinoside motif or hexa-arabinan motif, (iv) or one or more solutions for diluting a reagent in said kit.
